(19) United States
(12) Patent Application Publication
(10) Pub. No.: US 2009/0286727 A1
(43) Pub. Date: Nov. 19, 2009

(54) DP-78-Like Nanobodies

(75) Inventor: Joost Alexander Kolkman, Sint-Martens-Latem (BE)

Correspondence Address: WOLF GREENFIELD & SACKS, P.C., 600 ATLANTIC AVENUE, BOSTON, MA, 02210-2206, US

(73) Assignee: Ablynx N.V., Ghent-Zwijnaarde (BE)

(21) Appl. No.: 12/226,173

(22) PCT Filed: Apr. 12, 2007
(86) PCT No.: PCT/EP2007/003259

§ 371 Date: Feb. 23, 2009

Related U.S. Application Data
(60) Provisional application No. 60/792,279, filed on Apr. 14, 2006.

(51) Int. Cl. A61K 38/17; C12P 21/02; C12N 5/10; C40B 40/04; C07K 14/47; C07H 21/00

(52) U.S. Cl. 514/12; 435/69.1; 435/325; 506/15; 530/324; 536/23.1

(57) ABSTRACT

The present invention relates to Nanobodies® that have a high degree of sequence homology with human variable domain sequences from the V_{H}4 class and in particular with human DP-78 sequences, polypeptides containing such Nanobodies®, nucleic acids encoding such Nanobodies® and polypeptides, and uses thereof.

### SUMMARY OF THE INVENTION

The present invention relates to Nanobodies® that have a high degree of sequence homology with human variable domain sequences from the V_{H}4 class and in particular with human DP-78 sequences. [Note: Nanobody™, Nanobodies™ and Nanoclone™ are trademarks of Ablynx N. V.]

The invention also relates to polypeptides comprising such Nanobodies®, to nucleic acids encoding such Nanobodies® and polypeptides; to methods for preparing such Nanobodies® and polypeptides; to host cells expressing or capable of expressing such Nanobodies® or polypeptides; to compositions, and in particular to pharmaceutical compositions, that comprise such Nanobodies®, polypeptides, nucleic acids and/or host cells; and to uses of such Nanobodies®, polypeptides, nucleic acids, host cells and/or compositions, in particular for prophylactic, therapeutic or diagnostic purposes, such as the prophylactic, therapeutic or diagnostic purposes mentioned herein.

In one aspect of the invention, polypeptides comprising (an amino acid sequence that essentially consists of) four framework sequences and three complementarity determining sequences are provided. In the polypeptides, the framework sequences FR1 to FR4 (taken as a whole) have a degree of sequence identity with the framework sequences of the DP-78 sequence shown in FIG. 1 (SEQ ID NO:1) of more than 70%. In a preferred embodiment the degree of sequence identity is more than 80%. In a more preferred embodiment the degree of sequence identity is more than 85%. Still more preferably the degree of sequence identity is more than 90%, or even more than 95%. In no case will the framework sequences be 100% identical to SEQ ID NO:1. In another embodiment, the invention provides a polypeptide that comprises or essentially consists of at least one of the above described polypeptides.

In a further aspect of the invention, polypeptide including (an amino acid sequence that essentially consists of) four framework sequences and three complementarity determining sequences are provided. In the polypeptides, the framework sequences FR1 to FR4 (taken as a whole) have a degree of sequence identity with the framework sequences of the consensus V_{H}4 sequence of SEQ ID NO:6 of more than 70%. In a preferred embodiment the degree of sequence identity is more than 80%. In a more preferred embodiment the degree of sequence identity is more than 85%. Still more preferably the degree of sequence identity is more than 90%, or even more than 95%, and up to and including 100%. In another embodiment, the invention provides a polypeptide that comprises or essentially consists of one of the above described polypeptides.

Other aspects, embodiments, advantages and applications of the invention will become clear from the further description herein.

### BRIEF DESCRIPTION OF THE FIGURES

FIG. 1: Comparison of V_{H}4 and V_{H}3 sequences.

FIG. 2: Examples of V_{H}4 sequences.

FIG. 3: SDS-PAGE of purified Nanobodies of the invention (Example 4).

FIG. 4: Sensorgrams for V_{H}4 Nanobody binding to IL6 and IL6R (Example 6).

FIG. 5: Gel filtration profile of purified VH4 Nanobody 20.1 (Example 7).

FIG. 6: Alignment of the sequences of the V_{H}4 Nanobodies (Example 3)

FIG. 7: Alignment of the llama V_{H}4 V-gene sequences (Example 8).

### DETAILED DESCRIPTION OF THE INVENTION

For a description of so-called “heavy chain antibodies”, of the variable domains thereof, as well as Nanobodies® based thereon, reference is made to the following general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N. V. and Ablynx N. V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 WO 06/040153; WO 06/122786 and WO 06/122825 by Ablynx N. V. and the further published and unpublished patent applications by Ablynx N. V.; Hamers-Casterman et al., Nature 1993 Jun. 3; 363 (6428): 446-8; Davies and Riechmann, FEBS Lett. 1994 Feb. 21; 339(3): 285-90; Muyldermans et al., Protein Eng. 1994 September; 7(9): 1129-3; Davies and Riechmann, Biotechnology (NY) 1995 May; 13(5): 475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9(6): 531-7; Desmyter et al., Nat Struct Biol. 1996 September; 3(9): 803-11; Sheriff et al., Nat Struct Biol. 1996 September; 3(9): 733-6; Spinelli et al., Nat Struct Biol. 1996 September; 3(9): 752-7; Arbabi Ghahroudi et al., FEBS Lett. 1997 Sep. 15; 414(3): 521-6; Vu et al., Mol. Immunol. 1997 November-December; 34(16-17): 1121-31; Atarhouch et al., Journal of Camel Practice and Research 1997; 4: 177-182; Nguyen et al., J. Mol. Biol. 1998 Jan. 23; 275(3): 413-8; Lauwereys et al., EMBO J. 1998 Jul. 1; 17(13): 3512-20; Frenken et al., Res Immunol. 1998 July-August; 149(6):589-99; Transue et al., Proteins 1998 Sep. 1; 32(4): 515-22; Muyldermans and Lauwereys, J. Mol. Recognit. 1999 March-April; 12 (2): 131-40; van der Linden et al., Biochim. Biophys. Acta 1999 Apr. 12; 1431(1): 37-46.; Decanniere et al., Structure Fold. Des. 1999 Apr. 15; 7(4): 361-70; Ngyuen et al., Mol. Immunol. 1999 June; 36(8): 515-24; Woolven et al., Immunogenetics 1999 October; 50 (1-2): 98-101; Riechmann and Muyldermans, J. Immunol. Methods 1999 Dec. 10; 231 (1-2): 25-38; Spinelli et al., Biochemistry 2000 Feb. 15; 39(6): 1217-22; Frenken et al., J. Biotechnol. 2000 Feb. 28; 78(1): 11-21; Nguyen et al., EMBO J. 2000 Mar. 1; 19(5): 921-30; van der Linden et al., J. Immunol. Methods 2000 Jun. 23; 240 (1-2): 185-95; Decanniere et al., J. Mol. Biol. 2000 Jun. 30; 300 (1): 83-91; van der Linden et al., J. Biotechnol. 2000 Jul. 14; 80(3): 261-70; Harmsen et al., Mol. Immunol. 2000 August; 37(10): 579-90; Perez et al., Biochemistry 2001 Jan. 9; 40(1): 74-83; Conrath et al., J. Biol. Chem. 2001 Mar. 9; 276 (10): 7346-50; Muyldermans et al., Trends Biochem Sci. 2001 April; 26(4):230-5; Muyldermans S., J. Biotechnol. 2001 June; 74 (4): 277-302; Desmyter et al., J. Biol. Chem. 2001 Jul. 13; 276 (28): 26285-90; Spinelli et al., J. Mol. Biol. 2001 Aug. 3; 311 (1): 123-9; Conrath et al., Antimicrob Agents Chemother. 2001 October; 45 (10): 2807-12; Decanniere et al., J. Mol. Biol. 2001 Oct. 26; 313(3): 473-8; Nguyen et al., Adv Immunol. 2001; 79: 261-96; Muruganandam et al., FASEB J. 2002 February; 16 (2): 240-2; Ewert et al., Biochemistry 2002 Mar. 19; 41 (11): 3628-36; Dumoulin et al., Protein Sci. 2002 March; 11 (3): 500-15; Cortez-Retamozo et al., Int. J. Cancer. 2002 Mar. 20; 98 (3): 456-62; Su et al., Mol. Biol. Evol. 2002 March; 19 (3): 205-15; van der Vaart J M., Methods Mol. Biol. 2002; 178: 359-66; Vranken et al., Biochemistry 2002 Jul. 9; 41 (27): 8570-9; Nguyen et al., Immunogenetics 2002 April; 54 (1): 39-47; Renisio et al., Proteins 2002 Jun. 1; 47 (4): 546-55; Desmyter et al., J. Biol. Chem. 2002 Jun. 28; 277 (26): 23645-50; Ledeboer et al., J. Dairy Sci. 2002 June; 85 (6): 1376-82; De Genst et al., J. Biol. Chem. 2002 Aug. 16; 277 (33): 29897-907; Ferrat et al., Biochem. J. 2002 Sep. 1; 366 (Pt 2): 415-22; Thomassen et al., Enzyme and Microbial Technol. 2002; 30: 273-8; Harmsen et al., Appl. Microbiol. Biotechnol. 2002 December; 60 (4): 449-54; Jobling et al., Nat. Biotechnol. 2003 January; 21 (1): 77-80; Conrath et al., Dev. Comp. Immunol. 2003 February; 27 (2): 87-103; Pleschberger et al., Bioconjug. Chem. 2003 March-April; 14 (2): 440-8; Lah et al., J. Biol. Chem. 2003 Apr. 18; 278 (16): 14101-11; Nguyen et al., Immunology. 2003 May; 109 (1): 93-101; Joosten et al., Microb. Cell Fact. 2003 Jan. 30; 2 (1): 1; Li et al., Proteins 2003 Jul. 1; 52 (1): 47-50; Loris et al., Biol. Chem. 2003 Jul. 25; 278 (30): 28252-7; van Koningsbruggen et al., J. Immunol. Methods. 2003 August; 279 (1-2): 149-61; Dumoulin et al., Nature. 2003 Aug. 14; 424 (6950): 783-8; Bond et al., J. Mol. Biol. 2003 Sep. 19; 332 (3): 643-55; Yau et al., J. Immunol. Methods. 2003 Oct. 1; 281 (1-2): 161-75; Dekker et al., J. Virol. 2003 November; 77 (22): 12132-9; Meddeb-Mouelhi et al., Toxicon. 2003 December; 42 (7): 785-91; Verheesen et al., Biochim. Biophys. Acta 2003 Dec. 5; 1624 (1-3): 21-8; Zhang et al., J Mol. Biol. 2004 Jan. 2; 335 (1): 49-56; Stijlemans et al., J Biol. Chem. 2004 Jan. 9; 279 (2): 1256-61; Cortez-Retamozo et al., Cancer Res. 2004 Apr. 15; 64 (8): 2853-7; Spinelli et al., FEBS Lett. 2004 Apr. 23; 564 (1-2): 35-40; Pleschberger et al., Bioconjug. Chem. 2004 May-June; 15 (3): 664-71; Nicaise et al., Protein Sci. 2004 July; 13 (7): 1882-91; Omidfar et al., Tumour Biol. 2004 July-August; 25 (4): 179-87; Omidfar et al., Tumour Biol. 2004 September-December; 25(5-6): 296-305; Szynol et al., Antimicrob Agents Chemother. 2004 September; 48(9):3390-5; Saerens et al., J. Biol. Chem. 2004 Dec. 10; 279 (50): 51965-72; De Genst et al., J. Biol. Chem. 2004 Dec. 17; 279 (51): 53593-601; Dolk et al., Appl. Environ. Microbiol. 2005 January; 71(1): 442-50; Joosten et al., Appl Microbiol Biotechnol. 2005 January; 66(4): 384-92; Dumoulin et al., J. Mol. Biol. 2005 Feb. 25; 346 (3): 773-88; Yau et al., J Immunol Methods. 2005 February; 297 (1-2): 213-24; De Genst et al., J. Biol. Chem. 2005 Apr. 8; 280 (14): 14114-21; Huang et al., Eur. J. Hum. Genet. 2005 Apr. 13; Dolk et al., Proteins. 2005 May 15; 59 (3): 555-64; Bond et al., J. Mol. Biol. 2005 May 6; 348(3):699-709; Zarebski et al., J. Mol. Biol. 2005 Apr. 21.

In accordance with the terminology used in the above references, the variable domains present in naturally occurring heavy chain antibodies will also be referred to herein as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art above, Nanobodies® can generally be described as proteins that have some of the functional properties and structural features that are characteristic of naturally occurring V_{HH} domains. These properties make V_{HH} domains, Nanobodies® and polypeptides containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies® can function as a single, relatively small, functional antigen-binding structural unit, domain or protein. This distinguishes the V_{HH} domains and Nanobodies® from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies® as (single) antigen-binding proteins or as (single) antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
V_{HH} domains and Nanobodies® can be expressed from a single gene and require no post-translational folding or modifications;
V_{HH} domains and Nanobodies® can easily be engineered into multivalent and multispecific formats (as further discussed herein);
V_{HH} domains and Nanobodies® are highly soluble and do not have a tendency to aggregate (as with the mouse-derived antigen-binding domains described by Ward et al., Nature, Vol. 341, 1989, p. 544);
V_{HH} domains and Nanobodies® are highly stable to heat, pH, proteases and other denaturing agents or conditions (see for example Ewert et al., supra);
V_{HH} domains and Nanobodies® are easy and relatively cheap to prepare, even on a scale required for production. For example, V_{HH} domains, Nanobodies® and proteins/polypeptides containing the same can be produced using microbial fermentation (e.g. as further described below) and do not require the use of mammalian expression systems, as with for example conventional antibody fragments;
V_{HH} domains and Nanobodies®(D are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies® can show so-called cavity-binding properties (inter alia due to their extended CDR3 loop, compared to conventional V_{H} domains) and can therefore also access targets and epitopes not accessible to conventional 4-chain antibodies and antigen-binding fragments thereof. For example, it has been shown that V_{HH} domains and Nanobodies® can inhibit enzymes (see for example WO 97/49805; Transue et al., (1998); Lauwereys et al., (1998).

As also mentioned in the prior art above, Nanobodies® can either be naturally occurring V_{HH} domains, “humanized” V_{HH} domains or “camelized” V_{H} domains, as well as partially or fully synthetic proteins, as long as these proteins have (at least some of) the functional properties and structural features that are characteristic of naturally occurring V_{HH} domains. As also mentioned in the above prior art, Nanobodies® can also be formatted and used in multivalent and/or multispecific formats.

The Nanobodies® that have been described in the above prior art can—based on their sequence, but without any limitation—generally be divided into three groups, i.e.
a) The “GLEW-group”: Nanobodies® with the amino acid sequence GLEW at positions 44-47 according to the Kabat numbering and Q at position 108 according to the Kabat numbering. As further described herein, Nanobodies® within this group usually have a V at position 37, and can have a W, P, R or S at position 103, and preferably have a W at position 103. The GLEW group also comprises some GLEW-like sequences such as those mentioned in the footnotes to Tables A-2 to A-5 below;
b) The “KERE-group”: Nanobodies® with the amino acid sequence KERE or KQRE or a similar sequence at positions 43-46 according to the Kabat numbering and Q or L at position 108 according to the Kabat numbering. As further described herein, Nanobodies® within this group usually have a F at position 37, an L or F at position 47; and can have a W, P, R or S at position 103, and preferably have a W at position 103;
c) The “103 P, R, S-group”: Nanobodies® with a P, R or S at position 103. These Nanobodies® can have either the amino acid sequence GLEW (or a similar GLEW-type sequence) at positions 44-47 of the Kabat numbering or the amino acid sequence KERE or KQRE (or a similar KERE-type sequence) at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

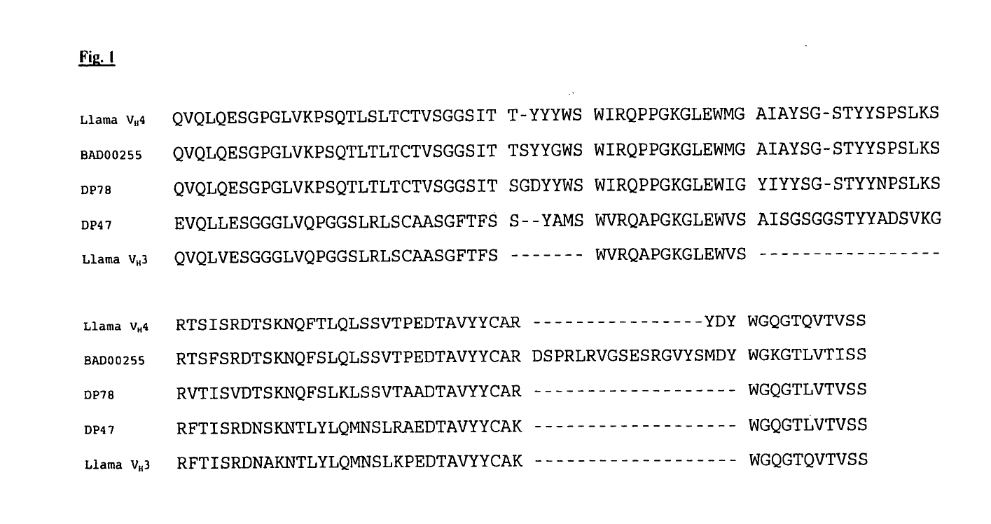

The known Nanobodies® from the GLEW group all have a high degree of sequence identity with the human germline sequence called DP-47. Reference is made to the sequence alignment shown in FIG. 1, in which the consensus sequence for the known GLEW-type Nanobodies® is indicated as the “Llama V_{H}3” sequence and the DP-47 germline sequence is indicated as “DP-47”.

Genbank entry BAD00255 (gi:38092356) describes a “immunoglobulin heavy chain VHDJ region” from Camelus dromedaries that has a high degree of sequence identity with DP-78. This sequence is shown in FIG. 1 as “BAD00255”. It is not clear from this entry whether this is a variable domain derived from a heavy chain antibodies or from a conventional 4-chain immunoglobulin (i.e. a V_{HH} domain or a V_{H} domain); however, the term “VHDJ region” seems to suggest that this a V_{H} domain instead of a V_{HH} domain. There is no mention of any antigen against which this sequence is directed, nor of any antigen binding activity or antigen binding specificity. Furthermore, this sequence has a framework 4 sequence which is ends on VTISS, whereas the

As will be clear from the above, all V_{HH} sequences and Nanobodies® of the GLEW-type disclosed in the art belong to the V_{H}3 class. It is therefore an object of the invention to provide a new class of Nanobodies® belonging to the GLEW-class, which are an alternative to the known llama V_{H}3 sequences.

Other objects of the invention will become clear from the further description herein.

It has now been found that the immune repertoire of Camelids (and in particular of llama glama) contains heavy chain antibodies that have variable domains that, without imposing any limitation, can be considered to belong to the V_{H}4 class or are related to the V_{H}4 class. In particular, it has now been found that the immune repertoire of Camelids (and in particular of llama glama) contains heavy chain antibodies that have variable domains that, without imposing any limitation, have a high degree of sequence identity with the human DP-78 germline sequence (shown in FIG. 1 as “DP-78”, and given in SEQ ID NO:1).

Thus, the invention generally provides isolated V_{HH} sequences (as well as Nanobodies based thereon or derived therefrom, as further defined herein) that, without imposing any limitation, can be considered to belong to the V_{H}4 class or are related to the V_{H}4 class. The invention also generally provides amino acid sequences/polypeptides that comprise, that essentially consist of and/or that are based on or derived from such V_{HH} sequences, which polypeptides are Nanobodies®, can be used as Nanobodies®, and/or can be used as a starting point for preparing or designing Nanobodies® (as further described herein).

More in particular, the invention provides isolated V_{HH} sequences (as well as Nanobodies based thereon or derived therefrom, as further defined herein) that, without imposing any limitation, have a high degree of sequence identity with the human germline sequence DP-78. The invention also generally provides Nanobodies® that comprise, that essentially consist of and/or that are based on or derived from such V_{HH} sequences. The VHH sequences and Nanobodies®(D disclosed herein have the favorable properties of the V_{HH} sequences and Nanobodies® described in the art. The V_{HH} sequences and Nanobodies® provided herein will also generally be referred to herein as “V_{H}4 sequences” or “V_{H}4-like Nanobodies®”.

Thus, the above objects are achieved by the Nanobodies® that are disclosed in the present specification and in the appended claims, in which:
a) Unless indicated or defined otherwise, all terms used have their usual meaning in the art, which will be clear to the skilled person. Reference is for example made to the standard handbooks, such as Sambrook et al., “Molecular Cloning: A Laboratory Manual” (2nd. Ed.), Vols. 1-3, Cold Spring Harbor Laboratory Press (1989); F. Ausubel et al., eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
b) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein;
d) Amino acid residues will be indicated according to the standard three-letter or one-letter amino acid code, as mentioned in Table A-1:

TABLE A-1
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residue can generally be considered essentially uncharged at a pH of about 6.5.

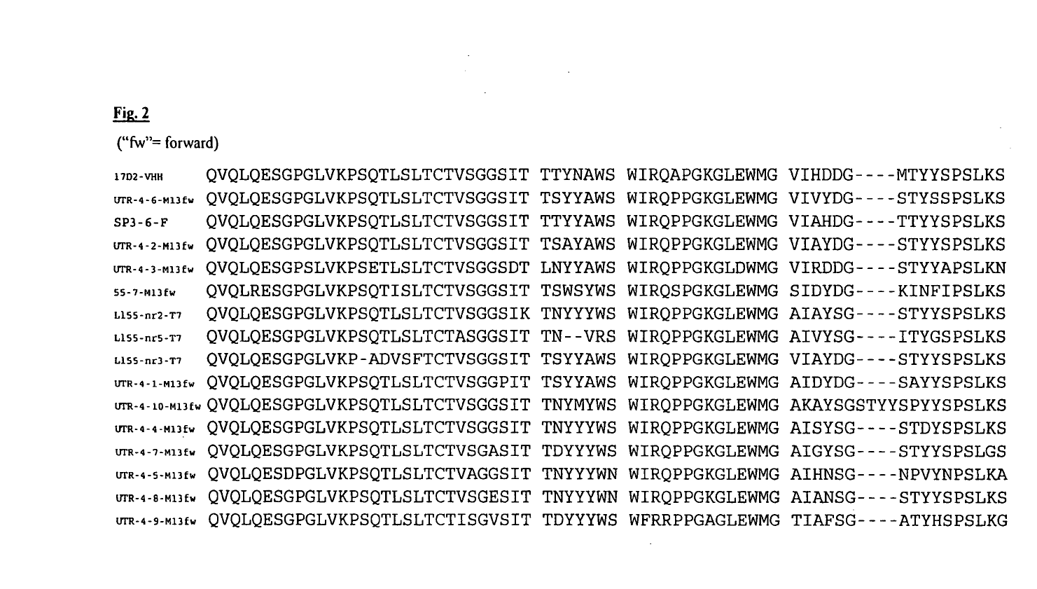

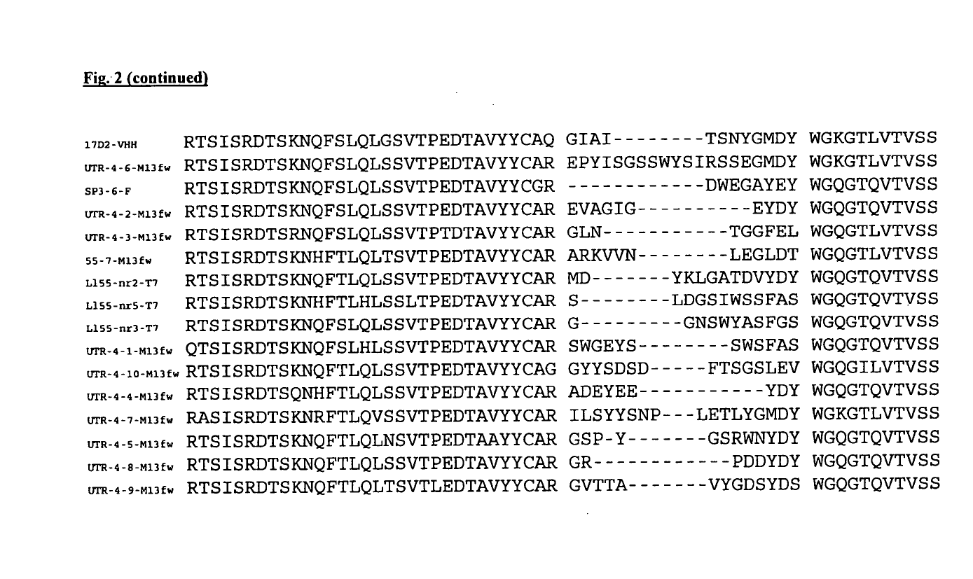

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).
   Alternatively, the degree of sequence identity between two or more nucleotide sequences may be calculated using a known computer algorithm for sequence alignment such as NCBI Blast v2.0, using standard settings.
   Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.
   Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of nucleotides in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.
   Alternatively, the degree of sequence identity between two amino acid sequences may be calculated using a known computer algorithm, such as those mentioned above for determining the degree of sequence identity for nucleotide sequences, again using standard settings.
   Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.
   Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.
   Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nat. Acad. Sci. USA 81: 140-144, 1984; Kyte & Doolittle; J. Molec. Biol. 157: 105-132, 1981, and Goldman et al., Ann. Rev. Biophys. Chem. 15: 321-353, 1986, all incorporated herein in their entirety by reference. Information on the primary, secondary and tertiary structure of Nanobodies® given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Nature Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions;
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
j) The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.
k) The term “antigenic determinant” refers to the epitope on the antigen recognized by the antigen-binding molecule (such as a Nanobody™ or a polypeptide of the invention) and more in particular by the antigen-binding site of said molecule. The terms “antigenic determinant” and “epitope’ may also be used interchangeably herein.
l) An amino acid sequence (such as a Nanobody™, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
m) The term “specificity” refers to the number of different types of antigens or antigenic determinants to which a particular antigen-binding molecule or antigen-binding protein (such as a Nanobody™ or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody™ or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the Nanobodies® and/or polypeptides of the invention) will bind with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ liters/mol is generally considered to indicate non-specific binding. Preferably, a Nanobody™ or polypeptide of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.
n) As further described herein, the amino acid sequence and structure of a Nanobody™ can be considered—without however being limited thereto—to be comprised of four framework regions or “FR's”, which are referred to in the art and herein as “Framework region 1” or “FR1 ”; as “Framework region 2” or “FR2”; as “Framework region 3” or “FR3”; and as “Framework region 4” or “FR4 ”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively;
o) As also further described herein, the total number of amino acid residues in a Nanobody™ can be in the region of 110-120, is preferably 112-115, and is most preferably 113. It should however be noted that parts, fragments, analogs or derivatives (as further described herein) of a Nanobody™ are not particularly limited as to their length and/or size, as long as such parts, fragments, analogs or derivatives meet the further requirements outlined herein and are also preferably suitable for the purposes described herein;
p) The amino acid residues of a Nanobody™ are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to herein (see for example FIG. 2 of said reference). According to this numbering, FR1 of a Nanobody™ comprises the amino acid residues at positions 1-30, CDR1of a Nanobody™ comprises the amino acid residues at positions 31-36, FR2of a Nanobody™ comprises the amino acids at positions 36-49, CDR2of a Nanobody™ comprises the amino acid residues at positions 50-65, FR3of a Nanobody™ comprises the amino acid residues at positions 66-94, CDR3of a Nanobody™ comprises the amino acid residues at positions 95-102, and FR4 of a Nanobody™ comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position I according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.).
   Alternative methods for numbering the amino acid residues of V_{H} domains, which methods can also be applied in an analogous manner to V_{HH} domains from Camelids and to Nanobodies®, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
q) The Figures and Sequence Listing are only given to further illustrate the invention and should not be interpreted or construed as limiting the scope of the invention and/or of the appended claims in any way, unless explicitly indicated otherwise herein.

Thus, in a first aspect, the invention relates to a polypeptide comprising (an amino acid sequence that essentially consists of) four framework sequences and three complementarity determining sequences, in which the framework sequences FR1 to FR4 (taken as a whole) have a degree of sequence identity (as defined herein) with the framework sequences of the DP-78 sequence shown in FIG. 1 (SEQ ID NO:1) of more than 70%, preferably more than 80%, even preferably more than 85%, such as more than 90% or even more than 95%, but not of 100%, and in which the complementarity determining sequences are as further described herein. In determining the degree of sequence identity for the purposes of the definition given in this paragraph, the sequence of the entire polypeptide of the invention can be compared to the sequence given in SEQ ID NO:1, in which—for determining the degree of sequence identity—any amino acid differences (as defined herein) at positions that form the complementarity determining sequences are disregarded (i.e. not taken into consideration). Generally, in the polypeptides according to this aspect of the invention, at least one of the framework sequences FR1 to FR4 will have at least one amino acid difference with the framework sequences FR1 to FR4 from the sequence of SEQ ID NO: 1. In this aspect of the invention, such an amino acid difference is preferably a “camelizing” amino acid difference (as described herein).

In another aspect, the invention relates to a polypeptide comprising (an amino acid sequence that essentially consists of) four framework sequences and three complementarity determining sequences, in which the amino acid sequence of each of the framework sequences FR1 to FR4 has no amino acid differences or 1 to 10 amino acid differences (as defined herein), and preferably 0 to 5 amino acid differences, such as 0, 1, 2, 3 or 4 amino acid differences, with the framework sequences FR1 to FR4 from the sequence of SEQ ID NO:1, respectively, and in which the complementarity determining sequences are as further described herein; provided that at least one of the framework sequences FR1 to FR4 has at least one amino acid difference with the framework sequences FR1 to FR4 the sequence of SEQ ID NO:1. In this aspect of the invention, such an amino acid difference is preferably a “camelizing” amino acid difference (as described herein).

For the purposes of determining the degree of sequence identity and/or the amino acid differences, the framework sequences of DP-78 are defined as follows (the numbering of the first and last amino acid residues in the sequence according to the Kabat numbering is added between brackets in italics). There are in total 87 amino acid residues in the framework sequence of DP-78:

[SEQ ID NO: 2]

FR1: [1] EVQLLESGGGLVQPGGSLRLSCAASGFTFS [30]

[SEQ ID NO: 3]

FR2: [36] WVRQAPGKGLEWVS [49]

[SEQ ID NO: 4]

FR3: [66] RFTISRDNSKNTLYLQMNSLRAEDTAVYYCAK [94](*)

[SEQ ID NO: 5]

FR4: [103] WGQGTLVTVSS [113]
(*) According w the Kabat numbering, the amino
acid residues “NSL” in FR3 are numbered “82a”
“82b” and “82c”, respectively, and the following
amino acid residues (“RAE” etc.) are numbered
“83”, “84”, “85”, etc.

Thus, in another aspect, the invention relates to a polypeptide comprising (an amino acid sequence that essentially consists of) four framework sequences and three complementarity determining sequences, in which:
FR1 has a degree of sequence identity with the amino acid sequence of SEQ ID NO:2 of more than 70%, preferably more than 80%, even more preferably more than 85%, such as more than 90% or even more then 95% and up to and including 100%;
FR2 has a degree of sequence identity with the amino acid sequence of SEQ ID NO:3 of more than 70%, preferably more than 80%, even more preferably more than 85%, such as more than 90% or even more then 95% and up to and including 100%;
FR3 has a degree of sequence identity with the amino acid sequence of SEQ ID NO:4 of more than 70%, preferably more than 80%, even more preferably more than 85%, such as more than 90% or even more then 95% and up to and including 100%;
FR4 has a degree of sequence identity with the amino acid sequence of SEQ ID NO:5 of more than 70%, preferably more than 80%, even more preferably more than 85%, such as more than 90% or even more then 95% and up to and including 100%;
   provided that at least one of the framework sequences FR1 to FR4 has at least one amino acid difference (as described herein) with the framework sequences SEQ ID NOs: 2 to 5, respectively. In this aspect of the invention, such an amino acid difference is preferably a “camelizing” amino acid difference (as described herein).

Thus, in another aspect, the invention relates to a polypeptide comprising four framework sequences and three complementarity determining sequences, in which:
FR1 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:2;
FR2 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:2;
FR3 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:2;
FR4 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:2;
   provided that at least one of the framework sequences FR1 to FR4 has at least one amino acid difference (as described herein) with the framework sequences SEQ ID NOs: 2 to 5, respectively. In this aspect of the invention, such an amino acid difference is preferably a “camelizing” amino acid difference (as described herein).

In the above aspects of the invention, the at least one amino acid difference between the polypeptide of the invention and the sequence of SEQ ID NO:1 is generally as defined herein and is most preferably such that the polypeptide of the invention has one or more of the favourable properties of Nanobodies® (as described herein) and/or can be used as a (single) antigen-binding protein or domain. Such an amino acid difference, which can be a substitution, deletion or insertion, and is preferably a substitution, is also referred to herein as a “camelizing” amino acid difference (as described herein). Preferred, but non-limiting examples of such camelizing amino acid differences will become clear from the disclosure herein, and based on this disclosure, the skilled person will be able to determine other suitable camelizing amino acid differences, optionally after a limited degree of routine experimentation. Generally, such camelizing amino acid differences will be at positions that, in the DP-78 sequence, form (part of) the V_{H}/V_{L} interface; and such positions will also become clear from the disclosure herein and/or can be determined by the skilled person based on the disclosure herein, optionally after a limited degree of routine experimentation.

Generally, such camelizing amino acid differences will be such that, compared to the DP-78 sequence, the ability of the amino acid residues that form the V_{H}/V_{L} interface (i.e. that would do so in DP-78) to undergo hydrophobic interactions with a V_{L} domain are reduced or inhibited. Examples of such camelizing amino acid differences (and in particular substitutions) will be clear to the skilled person, and may for example be the same or similar to the amino acid differences that are described in the prior art cited above for Nanobodies® from the V_{H}3 class (i.e. compared to human V_{H} sequences of the V_{H}3 class). For example, such a camelizing amino acid difference may comprise substitution of one or more of the amino acid residues in DP-78 (and in particular one or more of the amino acid difference that in DP-78 form part of the V_{H}/V_{L} interface) by a (more) polar or (more) charged amino acid residue, and in particular (more) charged amino acid residue, for which reference is made to Table A-1 above.

Some preferred, but non-limiting examples of camelizing amino acid differences will be clear to the skilled person from the V_{H}4 sequences of SEQ ID NO's 11-26 (mentioned in Table A-6 and as shown in FIG. 2), which are some non-limiting examples of the V_{H}4 sequences of the present invention.

The consensus sequence of the V_{H}4-like Nanobodies® is given in SEQ ID NO:6. The framework sequences from this consensus sequence are as follows (the numbering of the first and last amino acid residues in the sequence according to the Kabat numbering is added between brackets in italics. There are in total 87 amino acid residues in the four framework sequences):

[SEQ ID NO: 7]

FR1: [1] QVQLQESGPGLVKPSQTLSLTCTVSGGSIT [30]

[SEQ ID NO: 8]

FR2: [36] WIRQPPGKGLEWMG [49]

[SEQ ID NO: 9]

FR3: [66] RTSISRDTSKNQFTLQLSSVTPEDTAVYYCAR [94](*)

[SEQ ID NO: 10]

FR4: [103] WGQGTQVTVSS [113]
(*) According to the Kabat numbering (as applied
to Nanobodies by Riechmann and Muyldermans,
supra), the amino acid residues “NSL” in FR3 are
numbered “82a”, “82b” and “82c”, respectively, and
the following amino acid residues (“RAE” etc.) are
numbered “83”, “84”, “85”, etc.

Thus, in another aspect, the invention relates to a polypeptide comprising (an amino acid sequence that essentially consists of) four framework sequences and three complementarity determining sequences, in which the framework sequences FR1 to FR4 (taken as a whole) have a degree of sequence identity (as defined herein) with the framework sequences of the consensus V_{H}4 sequence of SEQ ID NO:6 of more than 70%, preferably more than 80%, even preferably more than 85%, such as more than 90% or even more than 95%, and up to and including 100%, and in which the complementarity determining sequences are as further described herein. In determining the degree of sequence identity for the purposes of the definition given in this paragraph, the sequence of the entire polypeptide of the invention can be compared to the sequence given in SEQ ID NO:6, in which—for determining the degree of sequence identity—any amino acid differences (as defined herein) at positions that form the complementarity determining sequences are disregarded (i.e. not taken into consideration). Generally, in the polypeptides according to this aspect of the invention, at least one of the framework sequences FR1 to FR4 will have at least one amino acid difference (and in particular at least one camelizing amino acid difference) with the framework sequences FR1 to FR4 from the sequence of SEQ ID NO:1.

In another aspect, the invention relates to a polypeptide comprising (an amino acid sequence that essentially consists of) four framework sequences and three complementarity determining sequences, in which the amino acid sequence of each of the framework sequences FR1 to FR4 has no amino acid differences or 1 to 10 amino acid differences (as defined herein), and preferably 0 to 5 amino acid differences, such as 0, 1, 2, 3 or 4 amino acid differences, with the framework sequences FR1 to FR4 from the sequence of SEQ ID NO:6, respectively, and in which the complementarity determining sequences are as further described herein. In this aspect of the invention, the amino acid difference may be any kind of amino acid difference (as generally defined herein), and may for example also be a humanizing amino acid difference (i.e. a humanizing insertion, deletion or substitution, and in particular a humanizing substitution). However, in the polypeptides according to this aspect of the invention, at least one of the framework sequences FR1 to FR4 has at least one amino acid difference (and in particular at least one camelizing amino acid difference) with the framework sequences FR1 to FR4 the sequence of SEQ ID NO:1.

Thus, in another aspect, the invention relates to a polypeptide comprising (an amino acid sequence that essentially consists of) four framework sequences and three complementarity determining sequences, in which:
FR1 has a degree of sequence identity with the amino acid sequence of SEQ ID NO:7 of more than 70%, preferably more than 80%, even more preferably more than 85%, such as more than 90% or even more then 95% and up to and including 100%;
FR2 has a degree of sequence identity with the amino acid sequence of SEQ ID NO:8 of more than 70%, preferably more than 80%, even more preferably more than 85%, such as more than 90% or even more then 95% and up to and including 100%;
FR3 has a degree of sequence identity with the amino acid sequence of SEQ ID NO:9 of more than 70%, preferably more than 80%, even more preferably more than 85%, such as more than 90% or even more then 95% and up to and including 100%;
FR4 has a degree of sequence identity with the amino acid sequence of SEQ ID NO:10 of more than 70%, preferably more than 80%, even more preferably more than 85%, such as more than 90% or even more then 95% and up to and including 100%;
   provided that at least one of the framework sequences FR1 to FR4 has at least one amino acid difference (and in particular at least one camelizing amino acid difference) with the framework sequences SEQ ID NOs: 2 to 5, respectively.

Thus, in another aspect, the invention relates to a polypeptide comprising (an amino acid sequence that essentially consists of) four framework sequences and three complementarity determining sequences, in which:
FR1 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:7;
FR2 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:8;
FR3 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:9;
FR4 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO:10.

In this aspect of the invention, the amino acid difference may be any kind of amino acid difference (as generally defined herein), and may for example also be a humanizing amino acid difference (i.e. a humanizing insertion, deletion or substitution, and in particular a humanizing substitution). However, in the polypeptides according to this aspect of the invention, at least one of the framework sequences FR1 to FR4 has at least one amino acid difference (and in particular at least one camelizing amino acid difference) with the framework sequences FR1 to FR4 the sequence of SEQ ID NO:1.

According to one preferred, but non-limiting aspect of the invention, the polypeptides described herein are such that they have, in at least one of the framework sequences FR1 to FR4, at least one amino acid difference (and in particular at least one camelizing amino acid difference) with the framework sequences FR1 to FR4, respectively, of a naturally occurring human V_{H}4 sequence

As mentioned above, the V_{H}4-like polypeptides of the present invention can generally be considered to belong to the GLEW-class of Nanobodies® (as described in the above prior art). This means that in the above polypeptides, positions 44-47 are GLEW or a “GLEW-like” sequence, such as for example GVEW, EPEW, GLER, DQEW, DLEW, GIEW, ELEW, GPEW, EWLP, GPER, GLER and ELEW or a similar sequence (as will be clear to the skilled person). More generally, and without limitation, a GLEW-type Nanobody can be described as a Nanobody in which position 44 is G and/or position 47 is W, and position 46 is usually E. Also, and again without limitation, in a GLEW-type Nanobody, position 45 is not a charged amino acid residue and not cysteine.

In addition, the polypeptides of the present invention preferably have at least any one, preferably at least any two, more preferably at least any three, such as at least any four, at least any five, at least any six, at least any seven or all of the following sequence characteristics (numbering according to Kabat numbering as applied by Riechmann and Muyldermans, above), which are some preferred, but non-limiting examples of the camelizing amino acid residues/amino acid differences of the present V_{H}4-like sequences:
position 30 is a T or K, preferably a T (e.g. compared to the human DP-78 sequence, where this position is an S); and/or
position 48 is an M (e.g. compared to the human DP-78 sequence, where this position is an I, and compared to the llama V_{H}3 sequence and the human DP-47, sequence, where this position is predominantly V); and/or
position 67 is a T (e.g. compared to the human DP-78 sequence, where this position is a V, and compared to the llama V_{H}3 sequence and the human DP-47, sequence, where this position is predominantly F); and/or
position 68 is a T (e.g. compared to the human DP-78 sequence, the llama V_{H}3 sequence and the human DP-47 sequence, where this position is predominantly T); and/or
position 71 is an R (e.g. compared to the human DP-78 sequence, where this position is a V); and/or
position 81 is a Q or H, and predominantly Q (e.g. compared to the human DP-78 sequence, where this position is predominantly K); and/or
position 83 is predominantly T; and/or
position 84 is predominantly P; and/or
position 85 is an E (e.g. compared to the human DP-78 sequence, where this position is predominantly A); and/or
at least two of the amino acid residues in CDR 1 (such as three of the amino acid residues in CDR1) are Y;
   or any suitable combination thereof.

Thus, for example, in a polypeptide of the invention, positions 66-70 are predominantly RTSIS, whereas in a human DP-78 sequence, these positions are predominantly RVTIS and in a llama V_{H}3 sequence and DP-47 sequence, these positions are predominantly RFTIS; and positions 83-85 are predominantly TPE, whereas in a human DP-78 sequence, these positions are predominantly TAA, in the llama V_{H}3 sequences, these positions are often KPE or EPE, and in a human DP-47 sequence, these positions are predominantly RAE.

Furthermore, compared to the llama V_{H}3 sequences and the human DP-47 sequences, the polypeptides of the invention may have one or more of the following sequence characteristics (which are also characteristic of the human DP-78 sequences compared to the llama V_{H}3 sequences and the human DP-47 sequences):
position 9 is a P (e.g. compared to the llama V_{H}3 sequences and the human DP-47, sequences, where this position is G); and/or
position 13 is a P (e.g. compared to the llama V_{H}3 sequences and the human DP-47, sequences, where this position is predominantly K); and/or
positions 15-17 are predominantly SQT (e.g. compared to the llama V_{H}3 sequences and the human DP-47, sequences, where these positions are predominantly GGS); and/or
position 19 is an S (e.g. compared to the llama V_{H}3 sequences and the human DP-47, sequences, where this position is predominantly R); and/or
position 21 is a T (e.g. compared to the llama V_{H}3 sequences and the human DP-47, sequences, where this position is predominantly A); and/or
position 24 is a V (e.g. compared to the llama V_{H}3 sequences and the human DP-47, sequences, where this position is predominantly A); and/or
position 23 is a T (e.g. compared to the llama V_{H}3 sequences and the human DP-47, sequences, where this position is predominantly A); and/or
position 24 is a V (e.g. compared to the llama V_{H}3 sequences and the human DP-47, sequences, where this position is predominantly A); and/or
position 37 is an I (e.g. compared to the llama V_{H}3 sequences, where this position is predominantly Y, and compared to the human DP-47 sequences, where this position is predominantly V); and/or
position 40 is a predominantly P (e.g. compared to the llama V_{H}3 sequences and the human DP-47 sequences, where this position is predominantly A) and/or
positions 73-74 are predominantly TS (e.g. compared to the llama V_{H}3 sequences and the human DP-47 sequences, where these positions are predominantly NA); and/or
positions 77-79 are predominantly QFT or QFS (e.g. compared to the llama V_{H}3 sequences, where these positions are predominantly TVY, and compared to the human DP-47 sequences, where these positions are predominantly SLY or TLY); and/or
position 82, 82a, 82b and 82c are predominantly LSSV (e.g. compared to the llama V_{H}3 sequences and the human DP-47 sequences, where these positions are predominantly MNSL); and/or
position 94 is predominantly R;
   or any suitable combination thereof.

In one specific, but non-limiting aspect of the invention, the polypeptide of the invention is as defined above, but is not BAD00255 (FIG. 1).

It should also be noted that amino acid sequences that essentially consist of four framework sequences and three complementarity determining sequences and that are as further defined herein form further aspects of the invention. Proteins and polypeptides that comprise or essentially consist of at least one (such as two, three, four or more) of such amino acid sequences (and optionally one or more further amino acid sequences, as further described herein) form another aspect of the invention. Also, when such a protein or polypeptide comprises two or more such amino acid sequences, and/or at least one such amino acid sequence and at least one further amino acid sequence, they may be suitably linked or fused to each other, either directly or via one or more suitable linkers (e.g. via covalent bonds, such as via chemical linkage or via genetic fusion). Also, in the invention, instead of a full-sized amino acid sequence as further defined herein, also one or more suitable fragments (also as further defined herein) may be used, and such fragments as well as proteins and polypeptides comprising or essentially consisting of one or more such fragments from further aspects of the invention.

As mentioned above, the V_{H}4-like sequences described herein can be used as such as Nanobodies®, and/or can be used as a starting point for preparing or designing (further) Nanobodies® (for example, without limitation, humanized Nanobodies®. Accordingly, in the present description, the polypeptides described herein (i.e. comprising an amino acid sequence that essentially consists of four framework sequences and three complementarity determining sequences that are as further defined herein) will also generally be referred to as “Nanobodies® of the invention” or more generally as “Nanobodies®”. However, unless explicitly mentioned herein, this nomenclature should not be interpreted as imposing any limitation on the origin, structure and/or properties of the polypeptides described herein. Thus, the polypeptides described herein for example generally encompass any binding domain or immunoglobulin sequence or fragment (including but not limited to the so-called “domain antibodies” or “single domain antibodies) that comprises four framework sequences and three complementarity determining sequences and that is further as defined herein. Thus, generally, the Nanobodies® as described herein can be of any origin, such as, without limitation, from natural origin (for example from mammalian origin such as from human origin or from Camelid origin), from synthetic origin or from semi-synthetic origin.

As mentioned above, the framework regions of the polypeptides of the present invention may contain one or more amino acid differences compared to the framework sequences of SEQ ID NOs: 7-10. Such amino acid differences may be any suitable amino acid differences that do not detract or detract too much from the favourable properties of the polypeptides described herein, and in particular from the favourable properties that are provided by the presence of the one or more camelizing amino acid residues (which camelizing residues and favourable properties are as described herein). The skilled person will be able to determine suitable amino acid differences based on the disclosure herein, optionally after a limited degree of routine experimentation. For example, such amino acid differences may comprise one or more conservative amino acid substitutions (as described herein). Other non-limiting examples of suitable amino acid differences (and in particular substitutions) will become clear from the further disclosure herein or will be clear to the skilled person from the prior art cited herein.

For example, in the Nanobodies® of the invention, one or more amino acid residues may be replaced by an amino acid residue that occurs at the corresponding position of a llama V_{H}3 sequence, as long as the resulting Nanobody™ retains at least one of the structural features mentioned above, and preferably also retains at least one, some and preferably all of the favourable properties of Nanobodies®. For this purpose, some preferred but-non limiting examples of amino acid residues that occur at the corresponding position of llama V_{H}3 sequences are mentioned in Tables A-2 to A-5 below.

Also, the Nanobodies® of the invention may also be (fully or partially) humanized, i.e. contain one or more “humanizing” amino acid differences (and in particular substitutions), in which for example one or more amino acid residues are replaced by amino acid residues that occur at the corresponding position of a human V_{H} sequence belonging to the DP-47 class or the DP-78 class, as long as the resulting Nanobody™ retains at least one of the structural features mentioned above, and preferably also retains at least one, some and preferably all of the favourable properties of Nanobodies®. For this purpose, some preferred but-non limiting examples of amino acid residues that occur at the corresponding position of a conventional DP-47 class or the DP-78 class are mentioned in Tables A-2 to A-5 below.

Preferably, in the humanized Nanobodies® of the invention, one or more amino acid residues are replaced by amino acid residues that occur at the corresponding position of a human V_{H} sequence belonging to the DP-78 class.

As for the Nanobodies® derived from llama V_{H}3 sequences, one particularly preferred, but non-limiting humanizing substitution is 108 Q to L.

It is also possible to combine two or more of the types of amino acid differences mentioned herein (e.g. to provide a Nanobody™ of the invention in which one or more amino acid residues have been replaced by an amino acid residue that occurs at the corresponding position of a llama V_{H}3 sequence and/or in which one or more amino acid residues have been replaced by an amino acid residue that occurs at the corresponding position of a human DP-78 V_{H} sequence and/or in which one or more amino acid residues have been replaced by an amino acid residue that occurs at the corresponding position of a human DP-47 V_{H} sequence), as long as the resulting Nanobody™ retains at least one of the structural features mentioned above, and preferably also retains at least one, some and preferably all of the favourable properties of Nanobodies®.

Also, as mentioned herein, the Nanobodies® of the invention (or nucleotide sequences encoding the same) may be provided by suitably “camelizing” a human V_{H} sequence, such as a human DP-78 sequence or other human V_{H}4 sequence.

In the context of the amino acid differences mentioned herein, it should be noted that in this context, terms such as “replaced by”, “replacing by”, “substituted” or “substitution” are generally meant to refer to an “amino acid difference” (as defined above) between two sequences at the indicated position, irrespective of how such an amino acid difference has been introduced or provided and irrespective of the origin of the sequences that are compared. Thus, these terms are not limited to providing an amino acid sequence (or a nucleotide sequence encoding the same) and replacing one amino acid residue by another amino acid residue (or by replacing, in said nucleotide sequence, a codon coding for one amino acid residue by a codon encoding another amino acid residue and then expressing the nucleotide sequence thus obtained), but also for example comprises, without limitation, amino acid sequences comprising such substitutions that have been obtained de novo by peptide synthesis (or by synthesis of a nucleotide sequence encoding such an amino acid sequence followed by expression of the same) and/or by suitably combining amino acid sequences derived from different Nanobody™ sequences and/or V_{H} sequences (and/or by suitably combining nucleotide sequences encoding such Nanobody™ and/or V_{H} sequences followed by expression of the combined nucleotide sequence thus obtained). Other suitable techniques for providing amino acid sequences containing the substitutions referred to herein (or for providing nucleotide sequences encoding the same) will be clear to the skilled person.

In the Tables A-2 to A-5 below, the consensus sequence for each of the sequences mentioned is indicated in bold.

TABLE A-2
Non-limiting examples of amino acid residues in FR1

| | Amino acid residue | | | |
|---|---|---|---|---|
| Pos. | Llama V_{H}4 | DP-78 | llama V_{H}3 | DP-47 |
| 1 | Q | Q | Q, A, E | E, Q |
| 2 | V | V, L | V | V |
| 3 | Q | Q | Q, K | Q |
| 4 | L | L | L | L |
| 5 | Q, R | Q | Q, E, L, V | V, L |
| 6 | E | E, Q | E, D, Q, A | E |
| 7 | S | S, W | S, F | S, T |
| 8 | G, D | G | G | G, R |
| 9 | P | P, A, S | G | G |
| 10 | G | G | G, D, R | G, V |
| 11 | L | L | Hallmark residue: | L, V; |
| | | | L, M, S, V, W; | predom- |
| | | | preferably L | inantly L |
| 12 | V | V, L | V, A | V, I |
| 13 | K | K | Q, E, K, P, R | Q, K, R |
| 14 | P | P | A, Q, A, G, P, S, | P |
| | | | T, V | |
| 15 | S ⁽*⁾ | S | G | G |
| 16 | Q, A, E | Q or E, D, G | G, A, E, D | G, R |
| 17 | T, D | T | S, F | S |
| 18 | L, I, V | L | L, V | L |
| 19 | S | S | R, K, L, N, S, T | R, K |
| 20 | L, F | L | L, F, I, V | L |
| 21 | T | T | S, A, F, T | S |
| 22 | C | C | C | C |
| 23 | T | T or A | A, D, E, P, S, T, V | A, T |
| 24 | V, A, I | V | A, I, L, S, T, V | A |
| 25 | S, A | S, Y | S, A, F, P, T | S |
| 26 | G | G | G, A, D, E, R, S, | G |
| | | | T, V | |
| 27 | G, A, E, V | G, Y | S, F, R, L, P, G, N, | F |
| 28 | S, P | S | N, T, E, D, S, I, R, | T |
| | | | A, G, R, F, Y | |
| 29 | I, D | I, F, V | F, L, D, S, I, G, V, | F, Y |
| | | | A | |
| 30 | T, K | S | N, S, E, G, A, D, | S, D, G |
| | | | M, T | |

⁽*⁾ May be deleted, in particular when positions 16 and 17 are A and D, respectively.

TABLE A-3
Non-limiting examples of amino acid residues in FR2

| | Amino acid residue | | | |
|---|---|---|---|---|
| Pos. | Llama V_{H}4 | DP-78 | llama V_{H}3 | DP-47 |
| 36 | W | W | W | W |
| 37 | I, F | I | Hallmark residue: | V, I, F; usually V |
| | | | F⁽¹⁾, H, I, L, Y or | |
| | | | V, preferably F⁽¹⁾ | |
| | | | or Y | |
| 38 | R | R | R | R |
| 39 | Q, R | Q | Q, H, P, R | Q |
| 40 | P, A, S | P, H | A, F, G, L, P, T, V | A |
| 41 | P | P | P, A, L, S | P, S, T |
| 42 | G | G | G, E | G |
| 43 | K, A | K | K, D, E, N, Q, R, | K |
| | | | T, V | |
| 44 | G | G | Hallmark residue: | G |
| | | | G⁽²⁾, E⁽³⁾, A, D, Q, | |
| | | | R, S, L; preferably | |
| | | | G⁽²⁾, E⁽³⁾ or Q; | |
| | | | most preferably | |
| | | | G⁽²⁾ or E⁽³⁾ | |
| 45 | L | L | Hallmark residue: | L |
| | | | L⁽²⁾, R⁽³⁾, C, I, L, | |
| | | | P, Q, V; preferably | |
| | | | L⁽²⁾ or R⁽³⁾ | |
| 46 | E, D | E | E, D, K, Q, V | E, V |
| 47 | W | W | Hallmark residue: | W, Y |
| | | | W⁽²⁾, L⁽¹⁾ or F⁽¹⁾, | |
| | | | A, G, I, M, R, S, V | |
| | | | or Y; preferably | |
| | | | W⁽²⁾, L⁽¹⁾, F⁽¹⁾ or R | |
| 48 | M | I | V, I, L | V |
| 49 | G | G | A, S, G, T, V | S, A, G |

TABLE A-4
Non-limiting examples of amino acid residues in FR3.

| | Amino acid residue | | | |
|---|---|---|---|---|
| Pos. | Llama V_{H}4 | DP-78 | llama V_{H}3 | DP-47 |
| 66 | R | R | R | R |
| 67 | T | V | F, L, V | F |
| 68 | S | T | T, A, N, S | T |
| 69 | I | I, M | I, L, M, V | I |
| 70 | S | S | S, A, F, T | S |
| 71 | R | V | R, G, H, I, L, K, | R |
| | | | Q, S, T, W | |
| 72 | D | D | D, E, G, N, V | D, E |
| 73 | T | T, K, R | N, A, D, F, I, K, L, | N, D, G |
| | | | R, S, T, V, Y | |
| 74 | S | S | A, D, G, N, P, S, | A, S |
| | | | T, V | |
| 75 | K, Q, R | K | K, A, E, K, L, N, | K |
| | | | Q, R | |
| 76 | N | N | N, D, K, R, S, T, | N, S |
| | | | Y | |
| 77 | Q, H, R | Q | T, A, E, I, M, P, S | S, T, I |
| 78 | F | F | V, L, A, F, G, I, M | L, A |
| 79 | T or S | S | Y, A, D, F, H, N, | Y, H |
| | | | S, T | |
| 80 | L | L | L, F, V | L |
| 81 | Q, H | K | Q, E, I, L, R, T | Q |
| 82 | L, V | L | M, I, L, V | M |
| 82a | S, G, T | S | N, D, G, H, S, T | N, G |
| 82b | S | S | S, N, D, G, R, T | S |
| 82c | V, L | V | L, P, V | L |
| 83 | T | T | Hallmark residue: | R or K; usually R |
| | | | R, K⁽⁵⁾, N, E⁽⁵⁾, G, | |
| | | | I, M, Q or T; | |
| | | | preferably K or R; | |
| | | | most preferably K | |
| 84 | P | A | Hallmark residue: | A, T, D; |
| | | | P⁽⁵⁾, A, D, L, R, S, | predominantly A |
| | | | T, V; preferably P | |
| 85 | E, T | A, V | E, D, G, Q | E, G |
| 86 | D | D | D | D |
| 87 | T | T | T, A, S | T, M |
| 88 | A | A | A, G, S | A |
| 89 | V | V | V, A, D, I, L, M, | V, L |
| | | | N, R, T | |
| 90 | Y | Y | Y, F | Y |
| 91 | Y | Y | Y, D, F, H, L, S, | Y, H |
| | | | T, V | |
| 92 | C | C | C | C |
| 93 | A, G | A | A, N, G, H, K, N, | A, K, T |
| | | | R, S, T, V, Y | |
| 94 | R, G, Q | R | A, V, C, F, G, I, | K, R, T |
| | | | K, L, R, S or T | |

TABLE A-5
Non-limiting examples of amino acid residues in FR4.

| | Amino acid residue | | | |
|---|---|---|---|---|
| Pos. | llama V_{H}4 | DP-78 | llama V_{H}3 | DP-47 |
| 103 | W | W | Hallmark residue: | W |
| | | | W⁽⁴⁾, P⁽⁶⁾, R⁽⁶⁾, S; | |
| | | | preferably W | |
| 104 | G | G | Hallmark residue: | G |
| | | | G or D; preferably | |
| | | | G | |
| 105 | Q, K | Q | Q, E, K, P, R | Q, R |
| 106 | G | G | G | G |
| 107 | T, I | T | T, A, I | T |
| 108 | Q | L | Hallmark residue: | L, M or T; |
| | | | Q, L⁽⁷⁾ or R; | predominantly L |
| | | | preferably Q or | |
| | | | L⁽⁷⁾ | |
| 109 | V | V | V | V |
| 110 | T | T | T, I, A | T |
| 111 | V | V | V, A, I | V |
| 112 | S | S | S, F | S |
| 113 | S | S | S, A, L, P, T | S |

### Notes to Tables A-2 to A-4 Above.

(1) In particular, but not exclusively, in combination with KERE or KQRE at positions 43-46.
(2) Usually as GLEW at positions 44-47.
(3) Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
(4) With both GLEW at positions 44-47 and KERE or KQRE at positions 43-46.
(5) Often as KP or EP at positions 83-84 of naturally occurring V_{HH} domains.
(6) In particular, but not exclusively, in combination with GLEW at positions 44-47.
(7) With the proviso that when positions 44-47 are GLEW, position 108 is always Q.
(8) The GLEW group also contains GLEW-like sequences at positions 44-47, such as for example GVEW, EPEW, GLER, DQEW, DLEW, GIEW, ELEW, GPEW, EWLP, GPER, GLER and ELEW.

Some non-limiting examples of V_{H}4 sequences of the present invention (SEQ ID NOs: 11-26) and their framework sequences (SEQ ID NOs 27-42 (FR1); SEQ ID NOs 43-58 (FR2); SEQ ID NOs: 59-74 and SEQ ID NOs 75-90 (FR4)) are mentioned in Table A-6 and shown in FIG. 2.

TABLE A-6
Non-limiting examples of V_{H}4 sequences and their framework sequences

| | | | | |
|---|---|---|---|---|
| Full sequence | Framework 1 | Framework 2 | Framework 3 | Framework 4 |

| | | | | | | | | | |
|---|---|---|---|---|---|---|---|---|---|
| SEQ | | SEQ | | SEQ | | SEQ | | SEQ | |
| ID | | ID | | ID | | ID | | ID | |
| NO | Clone | NO | Sequence | NO | Sequence | NO | Sequence | NO | Sequence |

| | | | | | | | | | |
|---|---|---|---|---|---|---|---|---|---|
| 11 | 17D2-VHH | 27 | QVQLQESGPGLVKPSQTL | 43 | WIRQAPGKGLE | 58 | RTSISRDTSKNQFSLQLG | 75 | WGKGTLVTVSS |
| | | | SLTCTVSGGSIT | | WMG | | SVTPEDTAVYYCAQ | | |
| 12 | UTR-4-6- | 28 | QVQLQESGPGLVKPSQTL | 44 | WIRQPPGKGLE | 60 | RTSISRDTSKNQFSLQLS | 76 | WGKGTLVTVSS |
| | M13fw | | SLTCTVSGGSIT | | WMG | | SVTPEDTAVYYCAR | | |
| 13 | SP3-6-F | 29 | QVQLQESGPGLVKPSQTL | 45 | WIRQPPGKGLE | 61 | RTSISRDTSKNQFSLQLS | 77 | WGQGTQVTVSS |
| | | | SLTCTVSGGSIT | | WMG | | SVTPEDTAVYYCGR | | |
| 14 | UTR-4-2- | 30 | QVQLQESGPGLVKPSQTL | 46 | WIRQPPGKGLE | 62 | RTSISRDTSKNQFSLQLS | 78 | WGQGTQVTVSS |
| | M13Fw | | SLTCTVSGGSIT | | WMG | | SVTPEDTAVYYCAR | | |
| 15 | UTR-4-3- | 31 | QVQLQESGPSLVKPSETL | 47 | WIRQPPGKGLD | 63 | RTSISRDTSRNQFSLQLS | 79 | WGQGTLVTVSS |
| | M13fw | | SLTCTVSGGSDT | | WMG | | SVTPTDTAVYYCAR | | |
| 16 | 55-7- | 32 | QVQLRESGPGLVKPSQTI | 48 | WIRQSPGKGLE | 64 | RTSISRDTSKNHFTLQLT | 80 | WGQGTLVTVSS |
| | M13fw | | SLTCTVSGGSIT | | WMG | | SVTPEDTAVYYCAR | | |
| 17 | L155-nr2- | 33 | QVQLQESGPGLVKPSQTL | 49 | WIRQPPGKGLE | 65 | RTSISRDTSKNQFTLQLS | 81 | WGQGTQVTVSS |
| | T7 | | SLTCTVSGGSIK | | WMG | | SVTPEDTAVYYCAR | | |
| 18 | L155-nr5- | 34 | QVQLQESGPGLVKPSQTL | 50 | WIRQPPGKGLE | 66 | RTSISRDTSKNHFTLHLS | 82 | WGQGTQVTVSS |
| | T7 | | SLTCTASGGSIT | | WMG | | SLTPEDTAVYYCAR | | |
| 19 | L155-nr3- | 35 | QVQLQESGPGLVKP- | 51 | WIRQPPGKGLE | 67 | RTSISRDTSKNQFSLQLS | 83 | WGQGTQVTVSS |
| | T7 | | ADVSFTCTVSGGSIT | | WMG | | SVTPEDTAVYYCAR | | |
| 20 | UTR-4-1- | 36 | QVQLQESGPGLVKPSQTL | 52 | WIRQPPGKGLE | 68 | QTSISRDTSKNQFSLHLS | 84 | WGQGTQVTVSS |
| | M13fw | | SLTCTVSGGPIT | | WMG | | SVTPEDTAVYYCAR | | |
| 21 | UTR-4-10- | 37 | QVQLQESGPGLVKPSQTL | 53 | WIRQPPGKGLE | 69 | RTSISRDTSKNQFTLQLS | 85 | WGQGILVTVSS |
| | M13fw | | SLTCTVSGGSIT | | WMG | | SVTPEDTAVYYCAG | | |
| 23 | UTR-4-7- | 39 | QVQLQESGPGLVKPSQTL | 55 | WIRQPPGKGLE | 71 | RASISRDTSKNRFTLQVS | 87 | WGKGTLVTVSS |
| | M13fw | | SLTCTVSGASIT | | WMG | | SVTPEDTAVYYCAR | | |
| 24 | UTR-4-5- | 40 | QVQLQESDPGLVKPSQTL | 56 | WIRQPPGKGLE | 72 | RTSIDRDTSKNQFTLQLN | 88 | WGQGTQVTVSS |
| | M13fw | | SLTCTVAGGSIT | | WMG | | SVTPEDTAAYYCAR | | |
| 25 | UTR-4-8- | 41 | QVQLQESGPGLVKPSQTL | 57 | WIRQPPGKGLE | 73 | RTSISRDTSKNQETLQLS | 89 | WGQGTQVTVSS |
| | M13fw | | SLTCTVSGESIT | | WMG | | SVTPEDTAVYYCAR | | |
| 26 | UTR-4-9- | 42 | QVQLQESGPGLVKPSQTL | 58 | WIRQPPGKGLE | 74 | RTSISRDTSKNQFTLQLT | 90 | WGQGTQVTVSS |
| | M13Fw | | SLTCTISGVSIT | | WMG | | SVTLEDTAVYYCAR | | |

Thus, in another aspect, the invention relates to a polypeptide comprising four framework sequences and three complementarity determining sequences, in which:
FR1 has a degree of sequence identity with at least one of the amino acid sequence of SEQ ID NOs: 27-42 of more than 70%, preferably more than 80%, even more preferably more than 85%, such as more than 90% or even more then 95% and up to and including 100%;
FR2 has a degree of sequence identity with at least one of the amino acid sequence of SEQ ID NOs: 43-58 of more than 70%, preferably more than 80%, even more preferably more than 85%, such as more than 90% or even more then 95% and up to and including 100%;
FR3 has a degree of sequence identity with at least one of the amino acid sequence of SEQ ID NO: 59-74 of more than 70%, preferably more than 80%, even more preferably more than 85%, such as more than 90% or even more then 95% and up to and including 100%;
FR4 has a degree of sequence identity with at least one of the amino acid sequence of SEQ ID NOs: 75-90 of more than 70%, preferably more than 80%, even more preferably more than 85%, such as more than 90% or even more then 95% and up to and including 100%;
   provided that at least one of the framework sequences FR1 to FR4 has at least one amino acid difference (as described herein) with the framework sequences SEQ ID NOs: 2 to 5, respectively. In this aspect of the invention, such an amino acid difference is preferably a “camelizing” amino acid difference (as described herein).

Thus, in another aspect, the invention relates to a polypeptide comprising four framework sequences and three complementarity determining sequences, in which:
FR1 has no amino acid differences or 1 to 10 amino acid differences (as defined herein), and preferably 0 to 5 amino acid differences, such as 0, 1, 2, 3 or 4 amino acid differences, with one of the amino acid sequence of SEQ ID NO: 2742;
FR2 has no amino acid differences or 1 to 10 amino acid differences (as defined herein), and preferably 0 to 5 amino acid differences, such as 0, 1, 2, 3 or 4 amino acid differences, with at least one of the amino acid sequence of SEQ ID NO: 43-58;
FR3 has no amino acid differences or 1 to 10 amino acid differences (as defined herein), and preferably 0 to 5 amino acid differences, such as 0, 1, 2, 3 or 4 amino acid differences, with at least one of the amino acid sequence of SEQ ID NO: 59-74;
FR4 has no amino acid differences or 1 to 10 amino acid differences (as defined herein), and preferably 0 to 5 amino acid differences, such as 0, 1, 2, 3 or 4 amino acid differences, with the amino acid sequence of SEQ ID NO: 75-90;
   provided that at least one of the framework sequences FR1 to FR4 has at least one amino acid difference (as described herein) with the framework sequences SEQ ID NOs: 2 to 5, respectively. In this aspect of the invention, such an amino acid difference is preferably a “camelizing” amino acid difference (as described herein).

In another aspect the invention relates to a polypeptide comprising four framework sequences and three complementarity determining sequences, in which the framework sequences FR1 to FR4 (taken as a whole) have a degree of sequence identity (as defined herein) with the framework sequences (taken as a whole) of at least one of the sequences of SEQ ID NOs: 11-26 of more than 70%, preferably more than 80%, even preferably more than 85%, such as more than 90% or even more than 95%, and up to and including 100%, and in which the complementarity determining sequences are as further described herein. Generally, in the polypeptides according to this aspect of the invention, at least one of the framework sequences FR1 to FR4 will have at least one amino acid difference with the framework sequences FR1 to FR4 from the sequence of SEQ ID NO:1. In this aspect of the invention, such an amino acid difference is preferably a “camelizing” amino acid difference (as described herein).

In another aspect, the invention relates to a polypeptide comprising four framework sequences and three complementarity determining sequences, in which the amino acid sequence of each of the framework sequences FR1 to FR4 has no amino acid differences or 1 to 10 amino acid differences (as defined herein), and preferably 0 to 5 amino acid differences, such as 0, 1, 2, 3 or 4 amino acid differences, with the framework sequences FR1 to FR4 from at least one of the sequences of SEQ ID NOs: 11-26, respectively, and in which the complementarity determining sequences are as further described herein; provided that at least one of the framework sequences FR1 to FR4 has at least one amino acid difference with the framework sequences FR1 to FR4 the sequence of SEQ ID NO:1. In this aspect of the invention, such an amino acid difference is preferably a “camelizing” amino acid difference (as described herein).

In the Nanobodies® of the present invention, the CDR's can be any suitable CDR sequences or combination of CDR sequences, as will be clear to the skilled person. In particular, the CDR sequences can be such that the Nanobody™ is capable of binding to a desired antigen, and preferably is capable of binding to a desired antigen with an affinity and/or specificity that is as further described herein.

According to one preferred, but non-limiting embodiment, at least two of the amino acid residues in CDR1 (such as three of the amino acid residues in CDR1) are Y.

For example, the CDR sequences can be naturally occurring CDR sequences, synthetic CDR sequences or semi-synthetic CDR sequences; or any combination thereof.

Also, the CDR sequences can be derived from a Camelid (e.g. from a Camelid immunized with the desired antigen) or can be derived from any other mammal, such as a mouse or rabbit. The CDR sequences can also be human CDR sequences, for example obtained by screening a naïve library of human antibodies or antibody fragments (for example a phage display library of human V_{H} fragments) for binders with affinity for the desired antigen.

Thus, as will be clear to the skilled person, the framework sequences of the Nanobodies® of the invention can be used as protein scaffolds for any desired CDR sequences, which may for example be grafted onto the framework sequences disclosed herein in order to provide a Nanobody™ of the invention, and the use of the V_{H}4 sequences and framework sequences disclosed herein for this purpose form a further aspect of the present invention.

It is also within the scope of the invention to provide a collection, set or library of Nanobodies® of the invention with different CDR sequences, which may for example comprise at least 2, preferably at least 10, more preferably at least 24, even more preferably at least 96, and up to 10², 10³, 10⁴, 10⁵, 10⁶ or 10⁷ or more different Nanobody™ sequences, and which may optionally be in the form of an expression library or another library format that is suitable for screening purposes (such as a phage display or yeast display library. Reference is for example made to the review article by Hoogenboom, Nature Biotechnology 2005 September; 23(9):1105-16 for examples of such formats and for methods of generating and screening such libraries). Such a collection, set or library of amino acid sequences as described herein, which forms another aspect of the present invention, may for example be present in a multi-well plate format, such as 24, 96, 354 or 512 well plates, or may be otherwise suitably arrayed, for example on a suitable plate or medium.

Other aspects of the invention are a set, collection or library of polypeptides as described herein, of nucleic acids as described herein, and or hosts (including viruses) or host cells as described herein. As will be clear to the skilled person, in one preferred but non-limiting aspect, the sets, collections or libraries described herein are preferably suitable for purposes of screening and selection, for example using one of the techniques described herein.

For example, in the above methods, the set, collection or library of nucleotide sequences encoding the amino acid sequences or polypeptides described herein may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational, or semi-empirical approaches such as computer modelling techniques or biostatics or datamining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library)), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Yet another technique for obtaining V_{HH} sequences or Nanobody sequences of the invention that are directed against a pre-determined target involves suitably immunizing (i.e. so as to raise an immune response and/or heavy chain antibodies directed against the target), a transgenic mammal that is capable of expressing immunoglobulin sequences (such as heavy chain antibodies) that contain at least one amino acid sequence (i.e. contain a V_{HH} sequence or Nanobody) as defined herein, obtaining a suitable biological sample from said transgenic mammal that contains (nucleic acid sequences encoding) said V_{HH} sequences or Nanobody sequences (such as a blood sample, serum sample or sample of B-cells), and then generating V_{HH} sequences directed against the target, starting from said sample, using any suitable technique known per se (such as any of the screening or selection methods described herein, or a hybridoma technique). For example, for this purpose, transgenic mice, methods and techniques that are similar to the transgenic mice and the further methods and techniques described in WO 02/085945, WO 04/049794 and WO 06/008548 and Janssens et al., Proc. Natl. Acad. Sci. USA. 2006 Oct. 10; 103(41):15130-5 can be used (where the mice express immunoglobulins that contain at least one amino acid sequence as described herein). Such methods, as well as transgenic mammals that express immunoglobulins that contain at least one amino acid sequence (i.e. at least one V_{H} domain, V_{HH} sequence or Nanobody) described herein, as well as biological materials and samples (such as egg cells, sperm cells, embryo's, samples of blood or of other biological fluids, cells or cell samples such as B-cells, as well as hybridoma cells, expression libraries of nucleotide sequences as generally described herein, etc.), form further aspects of the invention.

Another aspect of the invention relates to a method for providing a Nanobody™ of the invention, which method comprising grafting (or otherwise suitably linking or combining) at least one CDR sequence (such as a CDR1 sequence, a CDR2 sequence and a CDR3 sequence) onto one or more framework sequences of the V_{H}4 sequences as described herein (i.e. a FR1 sequence, a FR2 sequence, a FR3 sequence and a FR4 sequence) in a suitable manner so as to provide a Nanobody™ of the invention. Such grafting or linking may for example be performed in any manner known per se, such as by suitably linking one or more suitable amino acid sequences, but is usually either performed by linking one or more nucleotide sequences (e.g. encoding the framework sequences and the CDR's, respectively) so as to provide a nucleotide sequence that encodes the desired Nanobody™ sequence and then suitably expressing the nucleotide sequence thus obtained, and/or by de novo synthesis of all or part of such a nucleotide sequence followed by suitable expression.

In another aspect of the invention, the CDR sequences present in the Nanobodies® of the invention may be generated by suitably immunizing a mammal with the desired antigen and then generating immunoglobulin sequences (such as V_{H} sequences) directed against the desired antigen from for example a blood sample or B-cells obtained from said mammal. The CDR sequences present in said immunoglobulin sequences may then be determined (e.g. by sequencing) and grafted onto the framework sequences described herein to provide a Nanobody™ of the invention (i.e. essentially as described herein). The mammal may be any suitable mammal, such as a mouse or a Camelid (in which case the immunoglobulin sequence from which the CDR's are derived may be a V_{H} sequence or a V_{HH} sequence, including both V_{H}3 sequences as well as V_{H}4 sequences).

One specific technique that can be used to obtain the Nanobodies of the invention involves the use of the Nanoclone™ technique described in WO 06/079372. When applied to the present invention, this method generally involves isolating a sample of B-cells or an individual B-cell that expresses or is capable of expressing an immunoglobulin sequence that comprises an amino acid sequence as described herein (i.e. a V_{HH} domain or Nanobody), followed by obtaining said V_{HH} domain or Nanobody (or a nucleotide sequence or nucleic acid encoding the same) from said B-cell or sample. For example, for the latter purpose, a suitable PCR step can be used (again as generally described in WO 06/079372) using primers that specifically amplify nucleotide sequences that encode the desired amino acid sequence of the invention (such as the primer sequences described herein).

Of course, in the methods described above, the V_{H}4 sequences (or nucleotide sequences encoding the same) with the desired framework sequences (i.e. as described herein) and with the desired CDR's (as mentioned herein) may also be synthesized de novo using suitable techniques known per se. It is also possible to obtain the V_{H}4 sequences of the invention (or nucleotide sequences encoding the same) starting from V_{H}4 sequences that are not of Camelid origin (such as of human origin) and/or from V_{H}4 sequences that occur in conventional 4-chain antibodies (including but not limiting to human 4-chain antibodies and Camelid 4-chain antibodies), by suitably introducing one or more Camelizing substitutions as described herein in a manner known per se, so as to provide a Nanobody™ of the invention (or a nucleotide sequence encoding the same).

In addition, when the mammal used for immunization with the desired antigen is a Camelid, the Nanobodies® of the invention (or nucleotide sequences encoding the same) may also be isolated or generated as such starting from B-cells, blood or another suitable biological sample that is obtained from such a suitably immunized Camelid. This may generally be performed in a manner known per se for generating V_{HH} sequences from Camelids (for which reference is made to the prior art cited herein), but by selecting or generating V_{H}4 sequences instead of V_{H}3 sequences (as in the prior art cited above). Based on the information on V_{H}4 sequences provided herein, this will now be within the skill of the artisan.

The invention also relates to the V_{HH} sequences or Nanobody sequences (either as amino acid sequences or nucleotide sequences) that are obtained by the above methods (as well as the further methods described herein), or alternatively by a method that comprises the one of the above methods and in addition at least the steps of determining the nucleotide sequence or amino acid sequence of said V_{HH} sequence or Nanobody sequence; and of expressing or synthesizing said V_{HH} sequence or Nanobody sequence in a manner known per se, such as by expression in a suitable host cell or host organism or by chemical synthesis.

However, in addition to the use of such general methods and techniques for generating V_{HH} sequences, the present invention also provides some specific methods and techniques for generating the V_{H}4 sequences disclosed herein, which are based on the finding that the V_{H}4 sequences described herein are in Camelids usually associated with (i.e. preceded by) a specific leader sequence and 5′ UTR sequence. Thus, by use of a PCR or another suitable amplification technique in which at least one primer is used that is specific for either this leader sequence or this UTR (and at least one other suitable primer known per se), it is possible to identify and/or selectively amplify one or more V_{H}4 sequences as described herein. For the purpose of designing such a primer, the consensus nucleotide sequence of the 5′ UTR's is given in SEQ ID NO: 91 and the consensus nucleotide sequence for the leader sequences is given SEQ ID NO: 92

Thus, another aspect of the invention relates to a method for generating at least one V_{H}4 sequence as described herein, or a set or library of V_{H}4 sequences as described herein, which method comprises providing a template nucleic acid that has been derived from a Camelid and performing an amplification reaction using a primer pair in which the first primer (i.e. the “forward primer”) is capable of hybridizing with the sequence of SEQ ID NO: 91 or the sequence of SEQ ID NO: 92 under the conditions of the amplification reaction, and in which the second primer (i.e. the “reverse primer”) may be any suitable primer known per se for the amplification of immunoglobulin sequences and in particular of V_{HH} sequences, for which reference is made to the prior art cited herein. For example, a reverse primer as described in EP 0 368 684 may be used, or an oligo-dT primer as described in WO 03/054016 may be used. Optionally, after the amplification reaction, the one or more amplified V_{H}4 sequence(s) may be isolated and expressed. Alternatively, they may be cloned (e.g. in an expression vector) or inserted into another vector suitable for expression and/or screening (e.g. phages or phagemids) and screened for affinity or specificity against a desired antigen (all in a manner known per se and as further described herein).

A preferred, but non-limiting forward primer that can hybridize with the 5′ UTR sequence of SEQ ID NO: 91 is given in SEQ ID NO: 93 and a preferred, but non-limiting forward primer that can hybridize with the leader sequence of SEQ ID NO: 92 is given in SEQ ID NO: 94.

The template nucleic acid may be DNA or RNA (such as mRNA) and is in particular DNA (i.e. genomic DNA, cDNA or DNA that has been generated as part of an RT-PCR) and may for example be obtained from B-cells. In particular, for generating V_{H}4 sequences that are directed against a desired antigen, the template nucleic acid may be obtained from (B-cells or another suitable biological sample obtained from) a Camelid that has been suitably immunized with said antigen. Thus, for example, the amplification reaction may be performed on template nucleic acid that has been obtained from an individual B-cell that has been selected for expression of immunoglobulin sequences (and in particular of heavy chain antibodies or V_{HH} sequences) against the desired antigen (for example using the Nanoclone™ procedure described in the co-pending PCT application PCT/EP2005/011819 by Ablynx N. V.) and/or may be performed on template nucleic acid(s) that form(s) part of a pool of nucleic acid(s) obtained from B-cells. The latter may result in the amplification of a set, collection or library of V_{H}4 sequences as described herein (optionally in the form of a suitable expression library), which may be screened against a desired antigen (i.e. as outlined herein and in the prior art cited above) in order to provide one or more V_{H}4 sequences directed against said antigen. Alternatively, in the latter embodiment, the template nucleic acid(s) may be obtained from a Camelid that has not been immunized with a desired antigen in order to generate a naïve library of V_{H}4 sequences, which may again be screened in a manner known per se against the desired antigen.

The amino acid sequences of the leader sequences are given in SEQ ID NOs 95-97, and these leader sequences form further aspects of the invention. For example, it is envisaged that such leader sequences may be used in a manner known per se for the expression of a desired amino acid sequence (i.e. a protein or polypeptide, such as an immunoglobulin sequence) in a desired host cell (i.e. a mammalian cell or other suitable prokaryotic or eukaryotic host or host cell in which the leader sequence is operable) and/or for directing the secretion of a desired amino acid sequence from such a host or host cell (i.e. upon suitable expression thereof). Further aspects of the invention relate to genetic constructs comprising the leader sequences described herein (i.e. in which the leader sequence is operatively linked to a nucleotide sequence encoding a polypeptide to be expressed) or fusion proteins comprising such a leader sequence (i.e. in which the leader sequence is fused with an expressed amino acid sequence). Other potential uses and applications of the leader sequences described herein will be clear to the skilled person and form further aspects of the invention.

In addition to (the use of) the full-sized Nanobodies® of the invention as disclosed herein, the scope of the invention also comprises (the use of) use parts or fragments, or combinations of two or more parts or fragments, of the Nanobodies® of the invention as defined herein.

Generally, such parts or fragments of the Nanobodies® of the invention (including analogs thereof) have amino acid sequences in which, compared to the amino acid sequence of the corresponding full length Nanobody™ of the invention (or analog thereof), one or more of the amino acid residues at the N-terminal end, one or more amino acid residues at the C-terminal end, one or more contiguous internal amino acid residues, or any combination thereof, have been deleted and/or removed.

Such parts or fragments are preferably such that they are directed against a known or desired antigen and more preferably such that they can bind to such a known or desired antigen with an affinity and/or specificity that are as described herein.

Also, any such part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody™ of the invention.

Any part or fragment is preferably further such that it comprises at least one of CDR, such as at least of CDR1, CDR2 and/or CDR3. More preferably, any part or fragment is such that it comprises at least two CDR's (i.e. any two of CDR1, CDR2 and CDR3) and even more preferably all three CDR's of the corresponding full-sized Nanobody™ of the invention (i.e. suitably connected by framework sequences as disclosed herein or by parts or fragments thereof).

It is also possible to combine two or more of such parts or fragments (i.e. from the same or different Nanobodies® of the invention) to provide a Nanobody™ of the invention (or a further part or fragment thereof, as defined herein). It is for example also possible to combine one or more parts or fragments of a Nanobody™ of the invention with one or more parts or fragments of a human V_{H} domain (in particular, but not exclusively, a human DP-78 sequence) and/or with one or more parts of another Nanobody™ or V_{HH} sequence (such as, without limitation, a V_{H}3 sequence).

According to one preferred embodiment, the parts or fragments have a degree of sequence identity of at least 50%, preferably at least 60%, more preferably at least 70%, even more preferably at least 80%, such as at least 90%, 95% or 99% or more with the corresponding full-sized Nanobody™ of the invention.

The parts and fragments, and nucleic acid sequences encoding the same, can be provided and optionally combined in any manner known per se. For example, such parts or fragments can be obtained by inserting a stop codon in a nucleic acid that encodes a full-sized Nanobody™ of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody™ of the invention or by synthesizing such a nucleic acid in a manner known per se. Parts or fragments may also be provided using techniques for peptide synthesis known per se.

The Nanobodies® of the invention are preferably directed against a known or desired antigen. More in particular, the Nanobodies® of the invention preferably have a specificity and/or affinity for the desired or known antigen that is as described herein. Even more preferably, the CDR's that are present in the Nanobodies® of the invention are such that the Nanobodies® of the invention are directed against a known or desired antigen, and in particular have a specificity and/or affinity for the desired or known antigen that is as described herein.

Again, as generally described herein, Nanobodies® of the invention that are directed against a known or desired antigen can be obtained in any suitable manner known per se, which usually involves at least one step of screening with or against the desired antigen, for example of a library of suitable immunoglobulin sequences (e.g. a library of V_{HH} sequences or V_{H}4 sequences) or of a population of B-cells that express heavy chain antibodies (in which such libraries or B-cells are preferably obtained from a mammal, and in particular a Camelid, immunized with the desired antigen, although alternatively also a naive library or synthetic library may be used).

The antigen may be any suitable antigen, as will be clear to the skilled person. For diagnostic and/or pharmaceutical purposes, the antigen may be any suitable pharmaceutical target, which may for example be a target that is present in the circulation of the subject to be treated, may be a heterologous target (for example a target on a bacterium, virus or other pathogen) or may be expressed on the surface of at least one cell or tissue of the subject to be treated. It is generally envisaged that Nanobodies® of the invention may be generated against all antigens and targets for or against which conventional antibodies can be generated, and examples thereof will be clear to the skilled person. In particular, it is envisaged that Nanobodies® of the invention may be generated against all antigens and targets for or against which Nanobodies® of the V_{H}3 class can be generated, as will be clear to the skilled person from the prior art cited herein. Some non-limiting examples of antigens against which the Nanobodies® of the invention may be directed include, without limitation, tumor necrosis factor (TNF) alpha, Von Willebrand Factor, amyloid-beta, epidermal growth factor receptor (EGFR) and IL-6.

The Nanobodies® of the invention may be used in essentially the same or an analogous manner to the known V_{H}3 sequences, for which reference is again made to the prior art cited above. The specific uses and applications of a specific Nanobody™ of the invention will usually depend mainly on the antigen or target against which it is directed, as will be clear to the skilled person.

The invention in its broadest sense also comprises derivatives of the Nanobodies® of the invention. Such derivatives can generally be obtained by modification, and in particular by chemical and/or biological (e.g. enzymatical) modification, of the Nanobodies® of the invention and/or of one or more of the amino acid residues that form the Nanobodies® of the invention.

Examples of such modifications, as well as examples of amino acid residues within the Nanobody™ sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody™ of the invention, and in particular of one or more functional groups, residues or moieties that confer one or more desired properties or functionalities to the Nanobody™ of the invention. Example of such functional groups will be clear to the skilled person.

For example, such modification may comprise the introduction (e.g. by covalent binding or in any other suitable manner) of one or more functional groups that that increase the half-life, the solubility and/or the absorption of the Nanobody™ of the invention, that reduce the immunogenicity and/or the toxicity of the Nanobody™ of the invention, that eliminate or attenuate any undesirable side effects of the Nanobody™ of the invention, and/or that confer other advantageous properties to and/or reduce the undesired properties of the Nanobodies® and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody™ of the invention, or optionally via a suitable linker or spacer, as will again be clear to the skilled person.

One of the most widely used techniques for increasing the half-life and/or the reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody™ of the invention, a Nanobody™ of the invention may be modified so as to suitably introduce one or more cysteine residues for attachment of PEG, or an amino acid sequence comprising one or more cysteine residues for attachment of PEG may be fused to the N- and/or C-terminus of a Nanobody™ of the invention, all using techniques of protein engineering known per se to the skilled person.

Preferably, for the Nanobodies® and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody™ or polypeptide of the invention.

Yet another modification may comprise the introduction of one or more detectable labels or other signal-generating groups or moieties, depending on the intended use of the labelled Nanobody™. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as ³H, ¹²⁵I, ³²P, ³⁵S, ¹⁴C, ⁵¹Cr ³⁶Cl, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵Mn, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled Nanobodies® and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the Nanobody™ of the invention to another protein, polypeptide or chemical compound that is bound to the other half of the binding pair, i.e. through formation of the binding pair. For example, a Nanobody™ of the invention may be conjugated to biotin, and linked to another protein, polypeptide, compound or carrier conjugated to avidin or streptavidin. For example, such a conjugated Nanobody™ may be used as a reporter, for example in a diagnostic system where a detectable signal-producing agent is conjugated to avidin or streptavidin. Such binding pairs may for example also be used to bind the Nanobody™ of the invention to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the Nanobody™ of the invention.

For some applications, in particular for those applications in which it is intended to kill a cell that expresses the target against which the Nanobodies®(D of the invention are directed (e.g. in the treatment of cancer), or to reduce or slow the growth and/or proliferation such a cell, the Nanobodies® of the invention may also be linked to a toxin or to a toxic residue or moiety. Examples of toxic moieties, compounds or residues which can be linked to a Nanobody™ of the invention to provide—for example—a cytotoxic compound will be clear to the skilled person and can for example be found in the prior art cited above and/or in the further description herein. One example is the so-called ADEPT™ technology WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, such derivatives are such that they are directed against a known or desired antigen and more preferably such that they can bind to such a known or desired antigen with an affinity and/or specificity that are as described herein.

As mentioned above, the invention also relates to proteins or polypeptides that essentially consist of or comprise at least one Nanobody™ of the invention. By “essentially consist of” is meant that the amino acid sequence of the polypeptide of the invention either is exactly the same as the amino acid sequence of a Nanobody™ of the invention or corresponds to the amino acid sequence of a Nanobody™ of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody™.

Said amino acid residues may or may not change, alter or otherwise influence the (biological) properties of the Nanobody™ and may or may not add further functionality to the Nanobody™. For example, such amino acid residues:
a) can comprise an N-terminal Met residue, for example as result of expression in a heterologous host cell or host organism.
b) may form a signal sequence or leader sequence that directs secretion of the Nanobody™ from a host cell upon synthesis. Suitable secretory leader peptides will be clear to the skilled person, and may be as further described herein. Usually, such a leader sequence will be linked to the N-terminus of the Nanobody™, although the invention in its broadest sense is not limited thereto;
c) may form a sequence or signal that allows the Nanobody™ to be directed towards and/or to penetrate or enter into specific organs, tissues, cells, or parts or compartments of cells, and/or that allows the Nanobody™ to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (2004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising a Nanobody™ of the invention, as mentioned below;
d) may form a “tag”, for example an amino acid sequence or residue that allows or facilitates the purification of the Nanobody™, for example using affinity techniques directed against said sequence or residue. Thereafter, said sequence or residue may be removed (e.g. by chemical or enzymatical cleavage) to provide the Nanobody™ sequence (for this purpose, the tag may optionally be linked to the Nanobody™ sequence via a cleavable linker sequence or contain a cleavable motif). Some preferred, but non-limiting examples of such residues are multiple histidine residues, glutatione residues and a myc-tag such as AAAEQKLISEEDLNGAA [SEQ ID NO:31];
e) may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies® of the invention.

According to another embodiment, a polypeptide of the invention comprises a Nanobody™ of the invention, which is fused at its amino terminal end, at its carboxy terminal end, or both at its amino terminal end and at its carboxy terminal end to at least one further amino acid sequence, i.e. so as to provide a fusion protein comprising said Nanobody™ of the invention and the one or more further amino acid sequences. Such a fusion will also be referred to herein as a “Nanobody™ fusion”.

The one or more further amino acid sequence may be any suitable and/or desired amino acid sequences. The further amino acid sequences may or may not change, alter or otherwise influence the (biological) properties of the Nanobody™, and may or may not add further functionality to the Nanobody™ or the polypeptide of the invention. Preferably, the further amino acid sequence is such that it confers one or more desired properties or functionalities to the Nanobody™ or the polypeptide of the invention.

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005),

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody™ of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

The further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody™ of the invention is directed, or a different protein, polypeptide, antigen, antigenic determinant or epitope). For example, the further amino acid sequence may provide a second binding site that is directed against a serum protein (such as, for example, human serum albumin or another serum protein such as IgG), so as to provide increased half-life in serum. Reference is for example made to EP 0 368 684, WO 91/01743, WO 01/45746, WO 04/003019 and WO 06/122787 (in which various serum proteins are mentioned), the International application by Ablynx N. V. entitled “Nanobodies®™ against amyloid-beta and polypeptides comprising the same for the treatment of degenerative neural diseases such as Alzheimer's disease” (in which various other proteins are mentioned), as well as to Harmsen et al., Vaccine, 23 (41); 4926-42.

According to another embodiment, the one or more further amino acid sequences may comprise one or more parts, fragments or domains of conventional 4-chain antibodies (and in particular human antibodies) and/or of heavy chain antibodies. For example, although usually less preferred, a Nanobody™ of the invention may be linked to a conventional (preferably human) V_{H} or V_{L} domain or to a natural or synthetic analog of a V_{H} or V_{L} domain, again optionally via a linker sequence (including but not limited to other (single) domain antibodies, such as the dAb's described by Ward et al.).

The at least one Nanobody™ may also be linked to one or more (preferably human) CH₁, CH₂ and/or CH₃ domains, optionally via a linker sequence. For instance, a Nanobody™ linked to a suitable CH₁ domain could for example be used—together with suitable light chains—to generate antibody fragments/structures analogous to conventional Fab fragments or F(ab′)2 fragments, but in which one or (in case of an F(ab′)2 fragment) one or both of the conventional V_{H} domains have been replaced by a Nanobody™ of the invention. Also, two Nanobodies® could be linked to a CH₃ domain (optionally via a linker) to provide a construct with increased half-life in vivo.

According to one specific embodiment of a polypeptide of the invention, one or more Nanobodies® of the invention may linked to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more CH₂ and/or CH₃ domains of an antibody, such as from a heavy chain antibody (as described herein) and more preferably from a conventional human 4-chain antibody; and/or may form (part of) and Fc region, for example from IgG, from IgE or from another human Ig. For example, WO 94/04678 describes heavy chain antibodies comprising a Camelid V_{HH} domain or a humanized derivative thereof (i.e. a Nanobody™), in which the Camelidae CH₂ and/or CH₃ domain have been replaced by human CH₂ and CH₃ domains, so as to provide an immunoglobulin that consists of 2 heavy chains each comprising a Nanobody™ and human CH₂ and CH₃ domains (but no CH₁ domain), which immunoglobulin has the effector function provided by the CH₂ and CH₃ domains and which immunoglobulin can function without the presence of any light chains. Other amino acid sequences that can be suitably linked to the Nanobodies® of the invention so as to provide an effector function will be clear to the skilled person, and may be chosen on the basis of the desired effector function(s). Reference is for example made to WO 04/058820, WO 99/42077 and WO 05/017148, as well as the review by Holliger and Hudson, supra. Coupling of a Nanobody™ of the invention to an Fc portion may also lead to an increased half-life, compared to the corresponding Nanobody™ of the invention. For some applications, the use of an Fc portion and/or of constant domains (i.e. CH₂ and/or CH₃ domains) that confer increased half-life without any biologically significant effector function may also be suitable or even preferred. Other suitable constructs comprising one or more Nanobodies® and one or more constant domains with increased half-life in vivo will be clear to the skilled person, and may for example comprise two Nanobodies® linked to a CH3 domain, optionally via a linker sequence. Generally, any fusion protein or derivatives with increased half-life will preferably have a molecular weight of more than 50 kD, the cut-off value for renal absorption.

The further amino acid sequences may also form a signal sequence or leader sequence that directs secretion of the Nanobody™ or the polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the Nanobody™ or polypeptide of the invention to be directed towards and/or to penetrate or enter into specific organs, tissues, cells, or parts or compartments of cells, and/or that allows the Nanobody™ or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the “Peptrans” vectors mentioned above, the sequences described by Cardinale et al. and the amino acid sequences and antibody fragments known per se that can be used to express or produce the Nanobodies® and polypeptides of the invention as so-called “intrabodies”, for example as described in WO 94/02610, WO 95/22618, U.S. Pat. No. 7,004,940, WO 03/014960, WO 99/07414; WO 05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

For some applications, in particular for those applications in which it is intended to kill a cell that expresses the target against which the Nanobodies® of the invention are directed (e.g. in the treatment of cancer), or to reduce or slow the growth and/or proliferation such a cell, the Nanobodies® of the invention may also be linked to a (cyto)toxic protein or polypeptide. Examples of such toxic proteins and polypeptides which can be linked to a Nanobody™ of the invention to provide—for example—a cytotoxic polypeptide of the invention will be clear to the skilled person and can for example be found in the prior art cited above and/or in the further description herein. One example is the so-called ADEPT™ technology WO 03/055527.

According to one preferred, but non-limiting embodiment, said one or more further amino acid sequences comprise at least one further Nanobody™, so as to provide a polypeptide of the invention that comprises at least two, such as three, four, five or more Nanobodies®, in which said Nanobodies® may optionally be linked via one or more linker sequences (as defined herein). Generally, in such constructs, one or more Nanobodies® of the invention may be combined with one or more other Nanobodies® of the invention (e.g. V_{H}4 sequences) and/or with one or more other Nanobodies® (e.g. V_{H}3 sequences).

Polypeptides of the invention that comprise two or more Nanobodies®, of which at least one is a Nanobody™ of the invention, will also be referred to herein as “multivalent” polypeptides of the invention, and the Nanobodies® present in such polypeptides will also be referred to herein as being in a “multivalent format”. For example a “bivalent” polypeptide of the invention comprises two Nanobodies®, optionally linked via a linker sequence, whereas a “trivalent” polypeptide of the invention comprises three Nanobodies®, optionally linked via two linker sequences; etc.; in which at least one of the Nanobodies® present in the polypeptide, and up to all of the Nanobodies® present in the polypeptide, is/are a Nanobody™ of the invention.

In a multivalent polypeptide of the invention, the two or more Nanobodies® may be the same or different, and may be directed against the same antigen or antigenic determinant (for example against the same part(s) or epitope(s) or against different parts or epitopes) or may alternatively be directed against different antigens or antigenic determinants; or any suitable combination thereof. For example, a bivalent polypeptide of the invention may comprise (a) two identical Nanobodies™; (b) a first Nanobody™ directed against a first antigenic determinant of a protein or antigen and a second Nanobody™ directed against the same antigenic determinant of said protein or antigen which is different from the first Nanobody™; (c) a first Nanobody™ directed against a first antigenic determinant of a protein or antigen and a second Nanobody™ directed against another antigenic determinant of said protein or antigen; or (d) a first Nanobody™ directed against a first protein or antigen and a second Nanobody™ directed against a second protein or antigen (i.e. different from said first antigen). Similarly, a trivalent polypeptide of the invention may, for example and without being limited thereto. comprise (a) three identical Nanobodies™; (b) two identical Nanobody™ against a first antigenic determinant of an antigen and a third Nanobody™ directed against a different antigenic determinant of the same antigen; (c) two identical Nanobody™ against a first antigenic determinant of an antigen and a third Nanobody™ directed against a second antigen different from said first antigen; (d) a first Nanobody™ directed against a first antigenic determinant of a first antigen, a second Nanobody™ directed against a second antigenic determinant of said first antigen and a third Nanobody™ directed against a second antigen different from said first antigen; or (e) a first Nanobody™ directed against a first antigen, a second Nanobody™ directed against a second antigen different from said first antigen, and a third Nanobody™ directed against a third antigen different from said first and second antigen.

Polypeptides of the invention that contain at least two Nanobodies®, in which at least one Nanobody™ is directed against a first antigen or antigenic determinant and at least one Nanobody™ is directed against a second antigen or antigenic determinant, will also be referred to as “multispecific” polypeptides of the invention, and the Nanobodies® present in such polypeptides will also be referred to herein as being in a “multispecific format”. Thus, for example, a “bispecific” polypeptide of the invention is a polypeptide that comprises at least one Nanobody™ directed against a first antigen or antigenic determinant and at least one further Nanobody™ directed against a second antigen or antigenic determinant, whereas a “trispecific” polypeptide of the invention is a polypeptide that comprises at least one Nanobody™ directed against a first antigen or antigenic determinant, at least one further Nanobody™ directed against a second antigen or antigenic determinant and at least one further Nanobody™ directed against a third antigen or antigenic determinant; etc.

In addition, it is also within the scope of the invention that the polypeptides of the invention contain two or more Nanobodies® and one or more further amino acid sequences (as mentioned herein).

For multivalent and multispecific polypeptides containing one or more V_{HH} domains and their preparation, reference is also made to Conrath et al., J. Biol. Chem., Vol. 276, 10.7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of the invention can be found in the applications by Ablynx N. V. referred to herein.

One preferred, but non-limiting example of a multispecific polypeptide of the invention comprises at least one Nanobody™ of the invention and at least one Nanobody™ that provides for an increased half-life. Some preferred, but non-limiting examples of such Nanobodies® include Nanobodies® directed against serum proteins, such as human serum albumin, thyroxine-binding protein, (human) transferrin, fibrinogen, an immunoglobulin such as IgG, IgE or IgM, or one of the other serum proteins listed in WO 04/003019.

Preferably, however, such a Nanobody™ directed against a serum protein is a V_{H}4 sequence as described herein (i.e. a Nanobody™ of the invention) and Nanobodies®(D of the invention that provide for increased half-life (and polypeptides comprising the same, such as multispecific Nanobody™ constructs) form a further aspect of the invention. In particular, such a Nanobody™ of the invention may be directed against a (human) serum protein. For example, for experiments in mice, Nanobodies® against mouse serum albumin (MSA) can be used, whereas for pharmaceutical use, Nanobodies® against human serum albumin can be used.

Generally, any derivatives and/or polypeptides of the invention with increased half-life (for example pegylated Nanobodies® or polypeptides of the invention, multispecific Nanobodies® directed against a desired antigen and (human) serum albumin, or Nanobodies® fused to an Fc portion, all as described herein) have a half-life that is at least 1.5 times, preferably at least 2 times, such as at least 5 times, for example at least 10 times or more than 20 times, the half-life of the corresponding Nanobody™ of the invention.

Also, any derivatives or polypeptides of the invention with an increased half-life preferably have a half-life of more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Half-life can generally be defined as the time taken for the serum concentration of the polypeptide to be reduce by 50%, in vivo, for example due to degradation of the ligand and/or clearance or sequestration of the ligand by natural mechanisms. Methods for pharmacokinetic analysis and determination of half-life are familiar to those skilled in the art. Details may be found in Kenneth, A et al.: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2nd Rev. ex edition (1982).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

In the polypeptides of the invention, the one or more Nanobodies® and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody™ by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z} (such as for example the GS5, GS7, GS9, GS15 and GS30 linkers as described in the applications by Ablynx N. V. cited above), and hinge-like regions such as the hinge regions of naturally occurring heavy chain antibodies or similar sequences (such as described in WO 94/04678).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for the desired antigen. Based on the disclosure herein, the skilled person will be able to determine the optimal linker(s) for use in a specific polypeptide of the invention, optionally after on some limited routine experiments.

For example, in multivalent polypeptides of the invention that comprise Nanobodies® directed against a multimeric antigen (such as a multimeric receptor or other protein), the length and flexibility of the linker are preferably such that it allows each Nanobody™ of the invention present in the polypeptide to bind to the antigenic determinant on each of the subunits of the multimer. Similarly, in a multispecific polypeptide of the invention that comprises Nanobodies® directed against two or more different antigenic determinants on the same antigen (for example against different epitopes of an antigen and/or against different subunits of a multimeric receptor, channel or protein), the length and flexibility of the linker are preferably such that it allows each Nanobody™ to bind to its intended antigenic determinant. Again, based on the disclosure herein, the skilled person will be able to determine the optimal linker(s) for use in a specific polypeptide of the invention, optionally after on some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies® of the invention). For example, linkers containing one or more charged amino acid residues can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after on some limited routine experiments.

Finally, when two or more linkers are used in the polypeptides of the invention, these linkers may be the same or different. Again, based on the disclosure herein, the skilled person will be able to determine the optimal linkers for use in a specific polypeptide, of the invention, optionally after on some limited routine experiments.

Usually, for easy of expression and production, a polypeptide of the invention will be a linear polypeptide. However, the invention in its broadest sense is not limited thereto. For example, when a polypeptide of the invention comprises three of more Nanobodies®, “it is possible to link them use a linker with three or more “arms”, which each “arm” being linked to a Nanobody™, so as to provide a “star-shaped” construct. It is also possible, although usually less preferred, to use circular constructs.

The invention also comprises derivatives of the polypeptides of the invention, which may be essentially analogous to the derivatives of the Nanobodies® of the invention, i.e. as described herein.

The invention also comprises proteins or polypeptides that “essentially consist” of a polypeptide of the invention (in which the wording “essentially consist of” has essentially the same meaning as indicated hereinabove).

According to one embodiment of the invention, the Nanobodies® and polypeptides of the invention are in essentially isolated from, as defined herein.

The Nanobodies®, polypeptides and nucleic acids of the invention can be prepared in a manner known per se, as will be clear to the skilled person from the further description herein. For example, the Nanobodies® and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the Nanobodies®, polypeptides and nucleic acids include the methods and techniques described herein.

As will be clear to the skilled person, one particularly useful method for preparing a Nanobody™ and/or a polypeptide of the invention generally comprises the steps of:
the expression, in a suitable host cell or host organism (also referred to herein as a “host of the invention”) or in another suitable expression system of a nucleic acid that encodes said Nanobody™ or polypeptide of the invention (also referred to herein as a “nucleic acid of the invention”), optionally followed by:
isolating and/or purifying the Nanobody™ or polypeptide of the invention thus obtained.

In particular, such a method may comprise the steps of:
cultivating and/or maintaining a host of the invention under conditions that are such that said host of the invention expresses and/or produces at least one Nanobody™ and/or polypeptide of the invention; optionally followed by:
isolating and/or purifying the Nanobody™ or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody™ and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises
a) at least one nucleic acid of the invention; operably connected to
b) one or more regulatory elements, such as a promoter and optionally a suitable terminator;
   and optionally also
c) one or more further elements of genetic constructs known per se;
   in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 7,207,410, U.S. Pat. No. 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the Nanobody™ or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces, for example of Schizosaccharomryces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example derived a cell or cell line derived from a human, from the mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The Nanobodies® and polypeptides of the invention can also be introduced and expressed in one or more cells, tissues or organs of a multicellular organism, for example for prophylactic and/or therapeutic purposes (e.g. as a gene therapy). For this purpose, the nucleotide sequences of the invention may be introduced into the cells or tissues in any suitable way, for example as such (e.g. using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

For expression of the Nanobodies® in a cell, they may also be expressed as so-called or as so-called “intrabodies”, as for example described in WO 94/02610, WO 95/22618 and U.S. Pat. No. 7,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

For production, the Nanobodies® and polypeptides of the invention can for example also be produced in the milk of transgenic mammals, for example in the milk of rabbits, cows, goats or sheep (see for example U.S. Pat. No. 6,741,957, U.S. Pat. No. 6,304,489 and U.S. Pat. No. 6,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the Nanobodies® and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies® is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, Polypeptides of the invention suitable for pharmaceutical use can be prepared using techniques for peptide synthesis.

For production on industrial scale, preferred heterologous hosts for the (industrial) production of Nanobodies® or Nanobody™-containing protein therapeutics include strains of E. coli, Pichia pastoris, S. cerevisiae that are suitable for large scale expression/production/fermentation, and in particular for large scale pharmaceutical expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody™-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression.

Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast are usually leads to a glycosylation pattern that differs from human glycosylation. Nevertheless, it should be understood that all the foregoing host cells and expression systems can be used in the invention, depending on the desired Nanobody™ or protein to be obtained.

Thus, according to one non-limiting embodiment of the invention, the Nanobody™ or polypeptide of the invention is glycosylated. According to another non-limiting embodiment of the invention, the Nanobody™ or polypeptide of the invention is non-glycosylated.

According to one preferred, but non-limiting embodiment of the invention, the Nanobody™ or polypeptide of the invention is produced in a bacterial cell, in particular a bacterial cell suitable for large scale pharmaceutical production, such as cells of the strains mentioned above.

According to another preferred, but non-limiting embodiment of the invention, the Nanobody™ or polypeptide of the invention is produced in a yeast cell, in particular a yeast cell suitable for large scale pharmaceutical production, such as cells of the species mentioned above.

According to yet another preferred, but non-limiting embodiment of the invention, the Nanobody™ or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the Nanobodies® and the proteins of the invention, the Nanobodies® and proteins of the invention can be produced either intracellularly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic hosts cells are used, extracellular production is usually preferred since this considerably facilitates the further isolation and downstream processing of the Nanobodies® and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm. Another advantage is that correct disulfide bonds may form because the periplasm provides a more oxidative environment than the cytoplasm. Proteins overexpressed in E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular a Nanobody™ or a polypeptide of the invention, can be used.

Thus, according to one non-limiting embodiment of the invention, the Nanobody™ or polypeptide of the invention is a Nanobody™ or polypeptide that has been produced intracellularly and that has been isolated from the host cell, and in particular from a bacterial cell or from an inclusion body in a bacterial cell. According to another non-limiting embodiment of the invention, the Nanobody™ or polypeptide of the invention is a Nanobody™ or polypeptide that has been produced extracellularly, and that has been isolated from the medium in which the host cell is cultivated.

Some preferred, but non-limiting promoters for use with these host cells include,
for expression in E. coli: lac promoter (and derivatives thereof such as the lacUV5 promoter); arabinose promoter; left-(PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC1 (cytochrome c iso-1), GAPDH (glyceraldehydes-3-phosphate dehydrogenase); PGK1 (phosphoglycerate kinase), PYK1 (pyruvate kinase); regulated: GAL1, 10, 7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in Pichia pastoris: the AOX1 promoter (alcohol oxidase I)
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1α (hEF-1α) promoter from human, chimpanzee, mouse or rat; the SV40 early promoter; HIV-1 long terminal repeat promoter; β-actin promoter;
   Some preferred, but non-limiting vectors for use with these host cells include:
vectors for expression in mammalian cells: pMAMneo (Clontech, Mountain View, Calif.), pcDNA3 (Invitrogen, Carlsbad, Calif.), pMC1neo (Stratagene, La Jolla, Calif.), pSG5 (Stratagene, LA Jolla, Calif.), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and IZD35 (ATCC 37565), as well as viral-based expression systems, such as those based on adenovirus;
vectors for expression in bacterial cells: pET vectors (Novagen, San Diego, Calif.) and pQE vectors (Qiagen, Valencia, Calif.);
vectors for expression in yeast or other fungal cells: pYES2 (Invitrogen, Carlsbad, Calif.) and Pichia expression vectors (Invitrogen, Carlsbad, Calif.);
vectors for expression in insect cells: pBlueBacII (Invitrogen, Carlsbad, Calif.) and other baculovirus vectors
vectors for expression in plants or plant cells: for example vectors based on cauliflower mosaic virus or tobacco mosaic virus, suitable strains of Agrobacterium, or Ti-plasmid based vectors.
   Some preferred, but non-limiting secretory sequences for use with these host cells include:
for use in bacterial cells such as E. coli: PelB, Bla, OmpA, OmpC, OmpF, OmpT, StII, PhoA, PhoE, MalE, Lpp, LamB, and the like; TAT signal peptide, hemolysin C-terminal secretion signal
for use in yeast: α-mating factor prepro-sequence, phosphatase (pho1), invertase (Suc), etc.;
for use in mammalian cells: indigenous signal in case the target protein is of eukaryotic origin; murine Ig κ-chain V-J2-C signal peptide; etc.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

The invention also relates to applications and uses of the Nanobodies® and polypeptides of the invention. As mentioned above, the Nanobodies® and polypeptides of the invention may be used for any suitable purpose known per se for Nanobodies® and polypeptides comprising the same (i.e. for V_{H}3 sequences and polypeptides comprising the same), for which reference is made to the prior art cited above. For example, of the Nanobodies® and polypeptides of the invention may be used for diagnostic and/or therapeutic purposes, as well as cosmetic purposes, but also for chromatography or other purification techniques, or for analytical techniques.

The invention also relates to compositions or kit-of-parts that comprise at least one Nanobody™ or polypeptide of the invention, or at least one nucleic acid encoding the same. Such a composition or kit of parts may be any suitable composition or kit of parts, depending on its desired or intended use. Reference is again made to the prior art cited above. For example, such kit of parts may be a diagnostic kit, as described in the art cited herein for V_{H}3 sequences.

For pharmaceutical or therapeutic use, the polypeptides of the invention may generally be formulated as a pharmaceutical preparation or composition comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one Nanobody™ of the invention or at least one polypeptide of the invention and at least one suitable carrier, diluent or excipient (i.e. suitable for pharmaceutical use), and optionally one or more further active substances.

Generally, the Nanobodies® and polypeptides of the invention can be formulated and administered in any suitable manner known per se, for which reference is for example made to the general background art cited above (and in particular to WO 04/041862, WO 04/041863, WO 04/041865 and WO 04/041867) as well as to the standard handbooks, such as Remington's Pharmaceutical Sciences, 18^{th} Ed., Mack Publishing Company, USA (1990) or Remington, the Science and Practice of Pharmacy, 21th Edition, Lippincott Williams and Wilkins (2005).

For example, the Nanobodies® and polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

The Nanobodies® and polypeptides of the invention can also be administered using gene therapy methods of delivery. See, e.g., U.S. Pat. No. 5,399,346, which is incorporated by reference in its entirety. Using a gene therapy method of delivery, primary cells transfected with the gene encoding a Nanobody™ or polypeptide of the invention can additionally be transfected with tissue specific promoters to target specific organs, tissue, grafts, tumors, or cells and can additionally be transfected with signal and stabilization sequences for subcellularly localized expression.

Thus, the Nanobodies® and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the Nanobodies® and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the Nanobody™ or polypeptide of the invention. The percentage of the compositions and preparations may, of course, be varied and may conveniently be between about 2 to about 60% of the weight of a given unit dosage form. The amount of the Nanobody™ or polypeptide of the invention in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the Nanobodies® and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the Nanobodies® and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The Nanobodies® and polypeptides of the invention may also be administered intravenously or intraperitoneally by infusion or injection. Solutions of the Nanobodies® and polypeptides of the invention or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the Nanobodies® and polypeptides of the invention in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the Nanobodies® and polypeptides of the invention may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the Nanobodies® and polypeptides of the invention can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the Nanobodies® and polypeptides of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the Nanobodies® and polypeptides of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the Nanobodies® and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the Nanobodies® and polypeptides of the invention required for use in treatment will vary not only with the particular Nanobody™ or polypeptide selected but also with the route of administration, the nature of the condition being treated and the age and condition of the patient and will be ultimately at the discretion of the attendant physician or clinician. Also the dosage of the Nanobodies® and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

As mentioned above, Nanobodies® and polypeptides of the invention that are directed against a known or desired pharmaceutically relevant target may be used in the prevention, treatment and/or diagnosis of diseases and disorders associated with such a target. Thus, in another aspect, the invention relates to a method for the prevention and/or treatment of at least one disease that is associated with a particular target, said method comprising administering, to a subject in need thereof, a pharmaceutically active amount of a Nanobody™ of the invention or polypeptide of the invention directed against said target, and/or of a pharmaceutical composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention also relates to a method for the prevention and/or treatment of at least one disease or disorder that can be prevented and/or treated by administering a Nanobody™ or polypeptide of the invention to a patient, said method comprising administering, to a subject in need thereof, a pharmaceutically active amount of a Nanobody™ of the invention, of a polypeptide of the invention, and/or of a pharmaceutical composition comprising the same.

In another embodiment, the invention relates to a method for immunotherapy, and in particular for passive immunotherapy, which method comprises administering, to a subject suffering from or at risk of the diseases and disorders mentioned herein, a pharmaceutically active amount of a Nanobody™ of the invention, of a polypeptide of the invention, and/or of a pharmaceutical composition comprising the same.

In the above methods, the Nanobodies® and/or polypeptides of the invention and/or the compositions comprising the same can be administered in any suitable manner, depending on the specific pharmaceutical formulation or composition to be used. Thus, the Nanobodies® and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The Nanobodies® and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific Nanobody™ or polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more Nanobodies® and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific Nanobody™ and polypeptide of the invention to be used, the specific route of administration and the specific pharmaceutical formulation or composition used, the Nanobodies® and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single Nanobody™ or polypeptide of the invention will be used. It is however within the scope of the invention to use two or more Nanobodies® and/or polypeptides of the invention in combination.

The Nanobodies® and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are administered to be simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of a Nanobody™ or polypeptide of the invention that is directed against a desired pharmaceutically relevant target in the preparation of a pharmaceutical composition for prevention and/or treatment of at least one disease or disorder associated with said target.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

The invention also relates to the use of a Nanobody™ or polypeptide of the invention in the preparation of a pharmaceutical composition for the prevention and/or treatment of at least one disease or disorder that can be prevented and/or treated by administering a Nanobody™ or polypeptide of the invention to a patient.

Again, in such a pharmaceutical composition, the one or more Nanobodies® or polypeptides of the invention may also be suitably combined with one or more other active principles, such as those mentioned herein.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

### Example 1

### Library Construction

Nucleotide sequences encoding V_{H}4 Nanobodies were amplified from total RNA from 3 different llamas immunized with human IL6 in a one-step RT-PCR reaction using primers Rev_UTR2 and For_hinge IgG3. The resulting amplicons were used as template in a nested PCR reaction using the For_FR1 V_{H}4 specific primer containing a SfiI restriction site and Rev_VTVSS primer. Primer sequences are show in Table B-1. The PCR products were subsequently digested with SfiI and BstEII (naturally occurring in FR4) and ligated into the corresponding restriction sites of phagemid vector pAX50 to obtain a library after electroporation in Escherichia coli TG 1. The phagemid vector allows for production of phage particles, expressing the individual V_{H}4 Nanobodies as a fusion protein with the geneIII product.

TABLE B-1
primer sequences

| | | |
|---|---|---|
| primer | sequence 5′--> 3′ | SEQ ID NO: |

| | | |
|---|---|---|
| Rev_UTR2 | ACAGCTCTGTCCTCACACAGG | 98 |
| For_hinge | CCAGCTCCAAGTGTCCCAA | 99 |
| IgG3 | | |
| For_FR1 | TAGTTCTAAACGGCCCAGCCGGCCATGG | 100 |
| V_{H}4 | CCCAGGTGCAGCTGCAGGAGTCGG | |
| Rev_VTVSS | TGAGGAGACGGTGACCTG | 101 |

### Example 2

### Selections

Different concentrations between 0 and 1 ug/ml of biotinylated human IL6 were immobilized on magnetic streptavidin beads. Phage were added and incubated for 2 hours. Unbound phage were washed away and bound phage were eluted by addition of trypsin and 30 min incubation at 37° C. Eluted phage were allowed to infect exponentially growing TG1 cells and are then plated on ampicillin containing LB agar plates.

### Example 3

### Identification of IL6 Specific Nanobodies

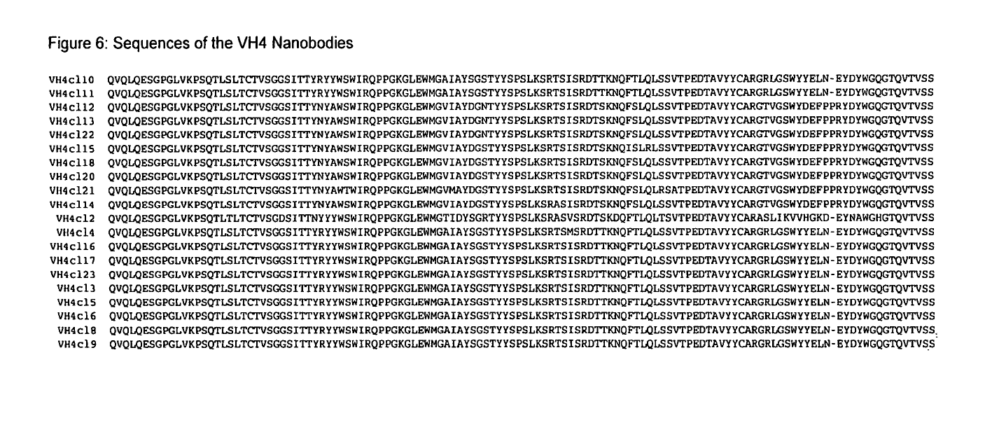

From the selection output where 1 ug/ml biotinylated human IL6 was used, 24 clones were picked and grown overnight in 2×YT+100 μg/ml ampicillin. After harvesting the cells, periplasmic extracts were prepared and analyzed for IL6 binding by ELISA. In this ELISA, 1 ug/ml bio-IL6 was immobilized in a neutravidin coated plate and periplasmic extracts were added in a 1/3 dilution. Bound Nanobodies were detected using anti-myc followed by GAM-HRP. ELISA results are shown in Table B-2. All 24 analyzed clones were sequenced and sequences are listed in Table B-3. An alignment of these sequences is shown in FIG. 6.

TABLE B-2
ELISA Results

| 5 | 6 | 7 |
|---|---|---|
| 0.1050 | 0.6470 | 0.6440 |
| 0.7550 | 0.7310 | 0.5000 |
| 0.5640 | 0.6870 | 0.3880 |
| 0.7930 | 0.5760 | 0.4930 |
| 0.8100 | 0.5810 | 0.4950 |
| 0.5320 | 0.5280 | 0.4970 |
| 0.1080 | 0.5110 | 0.6710 |
| 0.7050 | 0.6810 | 0.0990 |

### Example 4

### Small Scale Expression and Purification

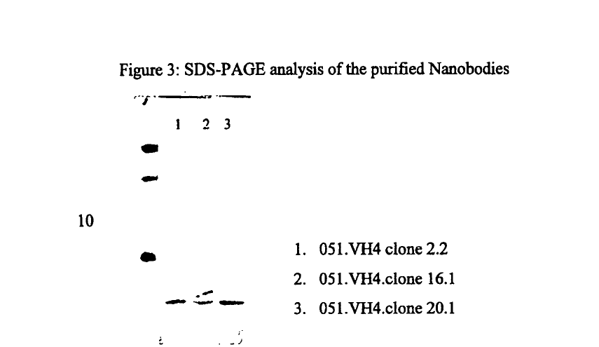

DNA fragments encoding 3 unique anti-IL6 V_{H}4 Nanobodies were digested with SfiI and BsteII, ligated into pAX51 vector and transformed into TG-1 competent cells. Carbenicillin resistant clones were analyzed for the presence of insert and sequences of positive clones were verified. TG-1 cells containing the V_{H}4 Nanobodies of interest were grown in TB medium+100 μg/ml Carbenicillin and induced by addition of IPTG for expression. The expression was allowed to continue for 4 hours. After collecting the cells, periplasmic extracts were prepared and the His6-tagged Nanobodies were purified by Immobilized Metal Affinity Chromatography (IMAC). Purified Nanobodies were dialyzed against PBS and concentrations were determined. 1 ug of each purified protein was analyzed by SDS-PAGE (FIG. 3).

### Example 5

### Biacore Analysis

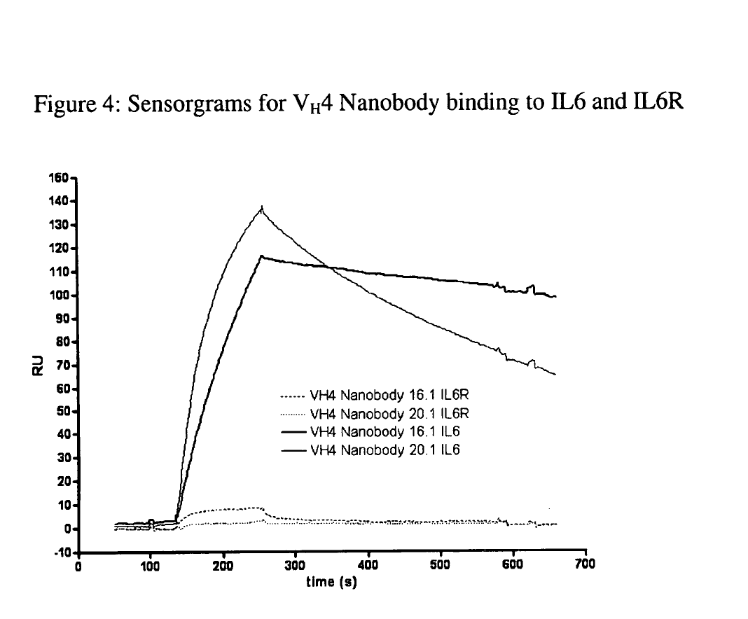

Binding of V_{H}4 Nanobodies to different antigens was evaluated by surface plasmon resonance on a Biacore 3000 instrument. Specificity of binding was analyzed by allowing 300 nM of V_{H}4 Nanobody to pass over a CM5 sensor chip containing either human IL6 or human IL6R. Sensorgrams of this experiment are shown in FIG. 4. Binding affinities for IL6 were determined by analyzing the association and dissociation phases at various concentrations of anti-IL6 V_{H}4 Nanobodies (ranging from 3.75 nM to 2 uM). Values for K_{d}, kₒₙ and k_{off} are given in Table B-4.

TABLE B-4
Affinity constants of Nanobodies of the invention

| Clone | kₒₙ (M⁻¹ · s⁻¹) | k_{off} (s⁻¹) | K_{d} (M) |
|---|---|---|---|
| V_{H}4 16.1 | 1.9E04 | 3.6E−04 | 1.9E−08 |
| V_{H}4 20.1 | 4.0E04 | 1.8E−03 | 4.6E−08 |

### Example 6

### Large Scale Expression

The DNA fragment encoding anti-IL6 V_{H}4 Nanobody 20.1 was cloned into the tagless pAX054 vector and then transformed into TG1 electrocompetent cells. Carbenicillin resistant clones were analyzed for the presence of insert and DNA sequences of positive clones were verified. Large scale expression of V_{H}4 Nanobody 20.1 was performed in a 10 liter bioreactor for approximately 18 hours. After centrifugation of the cell culture, the supernatant was used as starting material for the purification of the expressed Nanobody. The purification consists of several steps starting with 3 filtration steps followed by an anion exchange step on a Q Sepharose column. After acidification, the sample was loaded onto a S Sepharose column and the eluate was divided into 2 fractions. Fraction 1 was further purified using Source 30Q and Source S ion exchange columns and a final purification step on a SEC column. Fraction 2 was further purified using a source 30S column followed by 2 size exclusion steps. For both purification procedures pure V_{H}4 Nanobody (FIG. 4) was obtained. The yields were 3.2 mg and 2.0 mg from fraction 1 and 2, respectively.

### Example 7

### Analysis of Nanobody 20.1 by Analytical Gel Filtration

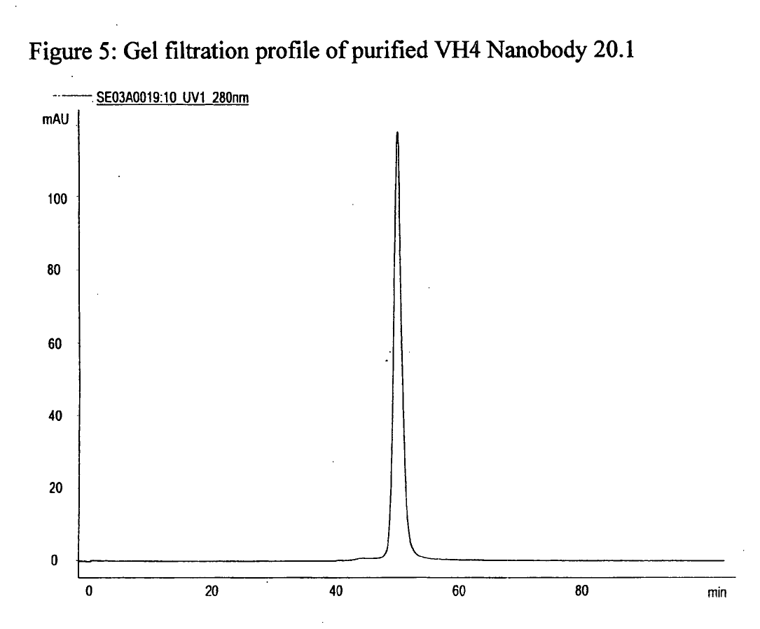

Approximately 10 ug of Nanobody 20.1 was applied to a TOSOH TSK-gel 62000SWXL gel-filtration column equilibrated in PBS (flow rate 0.2 ml/min). The chromatogram is shown in FIG. 5.

### Example 8

### Determination of Llama V_{H}4 V-Gene Sequences

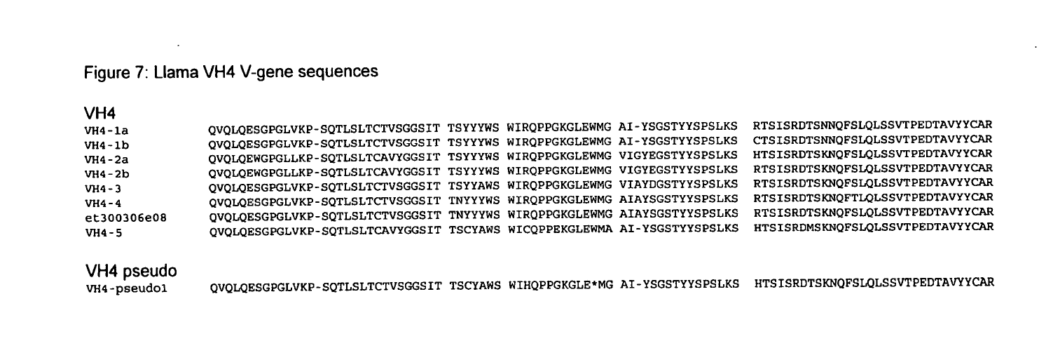

Genomic DNA isolated from testis of 2 llamas was used as template for PCR amplification. For each animal 3 PCR reactions were performed using UTR2 forward primer and 3 different RSS (Recombination Signal Sequence) specific reverse primers. Amplicons were cloned into the pCR4-TOPO vector and sequenced with M13rev primer. Altogether 53 readable sequences were obtained. Sequence analysis revealed the presence of 8 unique V_{H}4 gene segments and 1 pseudo gene. These sequences are listed in Table B-5. An alignment of these sequences in shown in FIG. 7.

TABLE B-3
Sequences of the V_{H}4 Nanobodies

V_{H}4 cl10 [SEQ ID NO: 102]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 Cl11 [SEQ ID NO: 103]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl12 [SEQ ID NO: 104]
QVQLQESGPGLVKPSQTLSLTCTVSGGSITTYNYAWSWIRQPPGKGLEWM
GVIAYDGNTYYSPSLKSRTSLSRDTSKNQFSLQLSSVTPEDTAVYYCARG
TVGSWYDEFPPRYDYWGQGTQVTVSS
V_{H}4 cl13 [SEQ ID NO: 105]
QVQLQESGPGLVKPSQTLSLTCTVSGGSITTYNYAWSWIRQPPGKGLEWM
GVIAYDGNTYYSPSLKSRTSISRDTSKNQFSLQLSSVTPEDTAVYYCARG
TVGSWYDEFPPRYDYWGQGTQVTVSS
V_{H}4 cl22 [SEQ ID NO: 106]
QVQLQESGPGLVKPSQTLSLTCTVSGGSITTYNYAWSWIRQPPGKGLEWM
GVIAYDGNTYYSPSLKSRTSISRDTSKNQFSLQLSSVTPEDTAVYYCARG
TVGSWYDEFPPRYDYWGQGTQVTVSS
V_{H}4 cl15 [SEQ ID NO: 107]
QVQLQESGPGLVKPSQTLSLTCTVSGGSITTYNYAWSWIRQPPGKGLEWM
GVIAYDGSTYYSPSLKSRTSISRDTSKNQISLRLSSVTPEDTAVYYCARG
TVGSWYDEFPPRYDYWGQGTQVTVSS
V_{H}4 cl18 [SEQ ID NO: 108]
QVQLQESGPGLVKPSQTLSLTCTVSGGSITTYNYAWSWIRQPPGKGLEWM
GVIAYDGSTYYSPSLKSRTSISRDTSKNQFSLQLSSVTPEDTAVYYCARG
TVGSWYDEFPPRYDYWGQGTQVTVSS
V_{H}4 cl20 [SEQ ID NO: 109]
QVQLQESGPGLVKPSQTLSLTCTVSGGSITTYNYAWSWIRQPPGKGLEWM
GVIAYDGSTYYSPSLKSRTSISRDTSKNQFSLQLSSVTPEDTAVYYCARG
TVGSWYDEFPPRYDYWGQGTQVTVSS
V_{H}4 cl21 [SEQ ID NO: 110]
QVQLQESGPGLVKPSQTLSLTCTVSGGSITTYNYAWTWIRQPPGKGLEWM
GVMAYDGSTYYSPSLKSRTSISRDTSKNQFSLQLRSATPEDTAVYYCARG
TVGSWYDEFPPRYDYWGQGTQVTVSS
V_{H}4 cl14 [SEQ ID NO: 111]
QVQLQESGPGLVKPSQTLSLTCTVSGGSITTYNYAWSWIRQPPGKGLEWM
GVIAYDGSTYYSPSLKSRASISRDTSKNQFSLQLSSVTPEDTAVYYCARG
TVGSWYDEFPPRYDYWGQGTQVTVSS
V_{H}4 cl2 [SEQ ID NO: 112]
QVQLQESGPGLVKPSQTLTLTCTVSGDSITTNYYYWSWIRQPPGKQLEWM
GTIDYSGRTYYSPSLKSRASVSRDTSKDQFTLQLTSVTPEDTAVYYCARA
SLIKVVHGKDEYNAWGHGTQVTVSS
V_{H}4 cl4 [SEQ ID NO: 113]
QVQLQESGPGLVKPSQTLSLTCTVSGGSITTYRYYWSWIRQPPGKGLEWM
GAIAYSGSTYYSPSLKSRTSMSRDTTKNQFTLQLSSVTPEDTAVYYCARG
RLGSWYYELNEYDYWGQGTQVTVSS
V_{H}4 cl16 [SEQ ID NO: 114]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl17 [SEQ ID NO: 115]
QVQLQESGPGLVKPSQTLSLTCTVSGOSFITYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl23 [SEQ ID NO: 116]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 Cl3 [SEQ ID NO: 117]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 Cl5 [SEQ ID NO: 118]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl6 [SEQ ID NO: 119]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl8 [SEQ ID NO: 120]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS
V_{H}4 cl9 [SEQ ID NO: 121]
QVQLQESGPGLVKPSQTLSLTCTVSGGSITTYRYYWSWIRQPPGKGLEWM
GAIAYSGSTYYSPSLKSRTSISRDTTKNQFTLQLSSVTPEDTAVYYCARG
RLGSWYYELNEYDYWGQGTQVTVSS

TABLE B-5
Llama V_{H}4 V-gene sequences

a) V_{H}4
V_{H}4-1a [SEQ ID NO: 122]
QVQLQESGPGLVKPSQTLSLTCTVSGGSITTSYYYWSWIRQPPGKGLEWM
GAIYSGSTYYSPSLKSRTSISRDTSNNQFSLQLSSVTPEDTAVYYCAR
V_{H}4-1b [SEQ ID NO: 123]
QVQLQESGPGLVKPSQTLSLTCTVSGGSITTSYYYWSWIRQPPGKGLEWM
GAIYSGSTYYSPSLKSCTSISRDTSNNQFSLQLSSVTPEDTAVYYCAR
V_{H}4-2a [SEQ ID NO: 124]
QVQLQEWGPGLLKPSQTLSLTCAVYGGSITTSYYYWSWIRQPPGKGLEWM
GVIGYEGSTYYSPSLKSHTSISRDTSKNQFSLQLSSVTPEDTAVYYCAR
V_{H}4-2b [SEQ ID NO: 125)
QVQLQEWGPGLLKPSQTLSLTCAVYGGSITTSYYYWSWIRQPPGKGLEWM
GVIGYEGSTYYSPSLKSRTSISRDTSKNQFSLQLSSVTPEDTAVYYCAR
V_{H}4-3 [SEQ ID NO: 126]
QVQLQESGPGLVKPSQTLSLTCTVSGGSITTSYYAWSWIRQPPGKGLEWM
GVIAYDGSTYYSPSLKSRTSISRDTSKNQFSLQLSSVTPEDTAVYYCAR
V_{H}4-4 [SEQ ID NO: 127]
QVQLQESGPGLVKPSQTLSLTCTVSGGSITTNYYYWSWIRQPPGKGLEWM
GAIAYSGSTYYSPSLKSRTSISRDTSKNQFTLQLSSVTPEDTAVYYCAR
et300306e08 [SEQ ID NO: 128]
QVQLQESGPGLVKPSQTLSLTCTVSGGSITTNYYYWSWIRQPPGKGLEWM
GAIAYSGSTYYSPSLKSRTSISRDTSKNQFSLQLSSVTPEDTAVYYCAR
V_{H}4-5 [SEQ ID NO: 129]
QVQLQESGPGLVKPSQTLSLTCAVYGGSITTSCYAWSWICQPPEKGLEWM
AAIYSGSTYYSPSLKSHTSISRDMSKNQFSLQLSSVTPEDTAVYYCAR
b) V_{H}4-pseudo
V_{H}4-pseudo 1 [SEQ ID NO: 130]
QVQLQESGPGLVKPSQTLSLTCTVSGGSITTSCYAWSWTHQPPGKGLEMG
AIYSGSTYYSPSLKSHTSISRDTSKNQFSLQLSSVTPEDTAVYYCAR

1. Amino acid sequence essentially consisting of four framework sequences and three complementarity determining sequences, in which the framework sequences FR1 to FR4 (taken as a whole) have a degree of sequence identity (as defined herein) with the framework sequences of the DP-78 sequence shown in FIG. 1 (SEQ ID NO:1) of more than 70%, preferably more than 80%, even preferably more than 85%, such as more than 90% or even more than 95%, but not of 100%.

2. Amino acid sequence essentially consisting of four framework sequences and three complementarity determining sequences, in which the framework sequences FR1 to FR4 (taken as a whole) have a degree of sequence identity (as defined herein) with the framework sequences of the consensus V_{H}4 sequence of SEQ ID NO: 6 of more than 70%, preferably more than 80%, even preferably more than 85%, such as more than 90% or even more than 95%, and up to and including 100%.

3. Amino acid sequence according to claim 1 or claim 2, in which the amino acid residue at position 44 according to the Kabat numbering is glycine (G) and/or in which the amino acid residue at position 47 according to the Kabat numbering is tryptophan (W).

4. Amino acid sequence according to claim 1 or claim 2, which is humanized.

5. Protein or polypeptide, comprising or essentially consisting of at least one amino acid sequence according to claim 1 or claim 2.

6. Protein or polypeptide comprising at least one amino acid sequence according to claim 1 or claim 2 and at least one further amino acid sequence, optionally linked via one or more suitable linkers.

7. Protein or polypeptide according to claim 5, which is a multivalent or multispecific protein or polypeptide.

8. Protein or polypeptide which comprises at least two polypeptides according to claim 1 or claim 2, optionally linked via one or more suitable linkers.

9. Nucleotide sequence or nucleic acid encoding an amino acid sequence according to claim 1 or claim 2.

10. Host cell or host organism that expresses or is capable of expressing an amino acid sequence according to claim 1 or claim 2.

11. Host cell or host organism that contains a nucleotide sequence or nucleic acid according to claim 9.

12. Set, collection or library of amino acid sequences according to claim 1 or claim 2.

13. Composition comprising at least one amino acid sequence according to claim 1 or claim 2.

14. Pharmaceutical composition comprising at least one amino acid sequence according to claim 1 or claim 2, and at least one pharmaceutically acceptable carrier, diluent or excipient and/or adjuvant, and optionally one or more further pharmaceutically active polypeptides and/or compounds.

15. Method for producing an amino acid sequence comprising at least the steps of:
expressing, in a suitable host cell or host organism or in another suitable expression system, a nucleic acid according to claim 9; optionally followed by:
isolating and/or purifying the amino acid sequence thus obtained.

16. Method for producing an amino acid sequence comprising at least the steps of:
cultivating and/or maintaining a host cell or host organism according to claim 10 under conditions that are such that said host cell expresses and/or produces the amino acid sequence; optionally followed by:
isolating and/or purifying the amino acid sequence thus obtained.

17. Protein or polypeptide according to claim 6, which is a multivalent or multispecific protein or polypeptide.


SEQUENCE LISTING (130 sequences)

SEQ ID NO: 1 - protein, 110 aa, Homo sapiens
QVQLQESGPGLVKPSQTLTLTCTVSGGSITSGDYYWSWIRQPPGKGLEWIGYIYYSGSTYYNPSLKSRVTISVDTSKNQFSLKLSSVTAADTAVYYCARWGQGTLVTVSS

SEQ ID NO: 2 - protein, 30 aa, Homo sapiens
EVQLLESGGGLVQPGGSLRLSCAASGFTFS

SEQ ID NO: 3 - protein, 14 aa, Homo sapiens
WVRQAPGKGLEWVS

SEQ ID NO: 4 - protein, 32 aa, Homo sapiens
RFTISRDNSKNTLYLQMNSLRAEDTAVYYCAK

SEQ ID NO: 5 - protein, 11 aa, Homo sapiens
WGQGTLVTVSS

SEQ ID NO: 6 - protein, 112 aa, Lama glama
QVQLQESGPGLVKPSQTLSLTCTVSGGSITTYYYWSWIRQPPGKGLEWMGAIAYSGSTYYSPSLKSRTSISRDTSKNQFTLQLSSVTPEDTAVYYCARYDYWGQGTQVTVSS

SEQ ID NO: 7 - protein, 30 aa, Lama glama
QVQLQESGPGLVKPSQTLSLTCTVSGGSIT

SEQ ID NO: 8 - protein, 14 aa, Lama glama
WIRQPPGKGLEWMG

SEQ ID NO: 9 - protein, 32 aa, Lama glama
RTSISRDTSKNQFTLQLSSVTPEDTAVYYCAR

SEQ ID NO: 10 - protein, 11 aa, Lama glama
WGQGTQVTVSS

SEQ ID NO: 11 - protein, 122 aa, Lama glama
QVQLQESGPGLVKPSQTLSLTCTVSGGSITTTYNAWSWIRQAPGKGLEWMGVIHDDGMTYYSPSLKSRTSISRDTSKNQFSLQLGSVTPEDTAVYYCAQGIAITSNYGMDYWGKGTLVTVSS

SEQ ID NO: 12 - protein, 130 aa, Lama glama
QVQLQESGPGLVKPSQTLSLTCTVSGGSITTSYYAWSWIRQPPGKGLEWMGVIVYDGSTYSSPSLKSRTSISRDTSKNQFSLQLSSVTPEDTAVYYCAREPYISGSSWYSIRSSEGMDYWGKGTLVTVSS

SEQ ID NO: 13 - protein, 118 aa, Lama glama
QVQLQESGPGLVKPSQTLSLTCTVSGGSITTTYYAWSWIRQPPGKGLEWMGVIAHDGTTYYSPSLKSRTSISRDTSKNQFSLQLSSVTPEDTAVYYCGRDWEGAYEYWGQGTQVTVSS

SEQ ID NO: 14 - protein, 120 aa, Lama glama
QVQLQESGPGLVKPSQTLSLTCTVSGGSITTSAYAWSWIRQPPGKGLEWMGVIAYDGSTYYSPSLKSRTSISRDTSKNQFSLQLSSVTPEDTAVYYCAREVAGIGEYDYWGQGTQVTVSS

SEQ ID NO: 15 - protein, 119 aa, Lama glama
QVQLQESGPSLVKPSETLSLTCTVSGGSDTLNYYAWSWIRQPPGKGLDWMGVIRDDGSTYYAPSLKNRTSISRDTSRNQFSLQLSSVTPTDTAVYYCARGLNTGGFELWGQGTLVTVSS

SEQ ID NO: 16 - protein, 122 aa, Lama glama
QVQLRESGPGLVKPSQTISLTCTVSGGSITTSWSYWSWIRQSPGKGLEWMGSIDYDGKINFIPSLKSRTSISRDTSKNHFTLQLTSVTPEDTAVYYCARARKVVNLEGLDTWGQGTLVTVSS

SEQ ID NO: 17 - protein, 123 aa, Lama glama
QVQLQESGPGLVKPSQTLSLTCTVSGGSIKTNYYYWSWIRQPPGKGLEWMGAIAYSGSTYYSPSLKSRTSISRDTSKNQFTLQLSSVTPEDTAVYYCARMDYKLGATDVYDYWGQGTQVTVSS

SEQ ID NO: 18 - protein, 120 aa, Lama glama
QVQLQESGPGLVKPSQTLSLTCTASGGSITTNVRSWIRQPPGKGLEWMGAIVYSGITYGSPSLKSRTSISRDTSKNHFTLHLSSLTPEDTAVYYCARSLDGSIWSSFASWGQGTQVTVSS

SEQ ID NO: 19 - protein, 120 aa, Lama glama
QVQLQESGPGLVKPADVSFTCTVSGGSITTSYYAWSWIRQPPGKGLEWMGVIAYDGSTYYSPSLKSRTSISRDTSKNQFSLQLSSVTPEDTAVYYCARGGNSWYASFGSWGQGTQVTVSS

SEQ ID NO: 20 - protein, 122 aa, Lama glama
QVQLQESGPGLVKPSQTLSLTCTVSGGPITTSYYAWSWIRQPPGKGLEWMGAIDYDGSAYYSPSLKSQTSISRDTSKNQFSLHLSSVTPEDTAVYYCARSWGEYSSWSFASWGQGTQVTVSS

SEQ ID NO: 21 - protein, 129 aa, Lama glama
QVQLQESGPGLVKPSQTLSLTCTVSGGSITTNYMYWSWIRQPPGKGLEWMGAKAYSGSTYYSPYYSPSLKSRTSISRDTSKNQFTLQLSSVTPEDTAVYYCAGGYYSDSDFTSGSLEVWGQGILVTVSS

SEQ ID NO: 22 - protein, 119 aa, Lama glama
QVQLQESGPGLVKPSQTLSLTCTVSGGSITTNYYYWSWIRQPPGKGLEWMGAISYSGSTDYSPSLKSRTSISRDTSQNHFTLQLSSVTPEDTAVYYCARADEYEEYDYWGQGTQVTVSS

SEQ ID NO: 23 - protein, 127 aa, Lama glama
QVQLQESGPGLVKPSQTLSLTCTVSGASITTDYYYWSWIRQPPGKGLEWMGAIGYSGSTYYSPSLGSRASISRDTSKNRFTLQVSSVTPEDTAVYYCARILSYYSNPLETLYGMDYWGKGTLVTVSS

SEQ ID NO: 24 - protein, 122 aa, Lama glama
QVQLQESDPGLVKPSQTLSLTCTVAGGSITTNYYYWNWIRQPPGKGLEWMGAIHNSGNPVYNPSLKARTSISRDTSKNQFTLQLNSVTPEDTAAYYCARGSPYGSRWNYDYWGQGTQVTVSS

SEQ ID NO: 25 - protein, 118 aa, Lama glama
QVQLQESGPGLVKPSQTLSLTCTVSGESITTNYYYWNWIRQPPGKGLEWMGAIANSGSTYYSPSLKSRTSISRDTSKNQFTLQLSSVTPEDTAVYYCARGRPDDYDYWGQGTQVTVSS

SEQ ID NO: 26 - protein, 123 aa, Lama glama
QVQLQESGPGLVKPSQTLSLTCTISGVSITTDYYYWSWFRRPPGAGLEWMGTIAFSGATYHSPSLKGRTSISRDTSKNQFTLQLTSVTLEDTAVYYCARGVTTAVYGDSYDSWGQGTQVTVSS

SEQ ID NO: 27 - protein, 30 aa, Lama glama; identical to SEQ ID NO: 7

SEQ ID NO: 28 - protein, 30 aa, Lama glama; identical to SEQ ID NO: 7

SEQ ID NO: 29 - protein, 30 aa, Lama glama; identical to SEQ ID NO: 7

SEQ ID NO: 30 - protein, 30 aa, Lama glama; identical to SEQ ID NO: 7

SEQ ID NO: 31 - protein, 30 aa, Lama glama
QVQLQESGPSLVKPSETLSLTCTVSGGSDT

SEQ ID NO: 32 - protein, 30 aa, Lama glama
QVQLRESGPGLVKPSQTISLTCTVSGGSIT

SEQ ID NO: 33 - protein, 30 aa, Lama glama
QVQLQESGPGLVKPSQTLSLTCTVSGGSIK

SEQ ID NO: 34 - protein, 30 aa, Lama glama
QVQLQESGPGLVKPSQTLSLTCTASGGSIT

SEQ ID NO: 35 - protein, 29 aa, Lama glama
QVQLQESGPGLVKPADVSFTCTVSGGSIT

SEQ ID NO: 36 - protein, 30 aa, Lama glama
QVQLQESGPGLVKPSQTLSLTCTVSGGPIT

SEQ ID NO: 37 - protein, 30 aa, Lama glama; identical to SEQ ID NO: 7

SEQ ID NO: 38 - protein, 30 aa, Lama glama; identical to SEQ ID NO: 7

SEQ ID NO: 39 - protein, 30 aa, Lama glama
QVQLQESGPGLVKPSQTLSLTCTVSGASIT

SEQ ID NO: 40 - protein, 30 aa, Lama glama
QVQLQESDPGLVKPSQTLSLTCTVAGGSIT

SEQ ID NO: 41 - protein, 30 aa, Lama glama
QVQLQESGPGLVKPSQTLSLTCTVSGESIT

SEQ ID NO: 42 - protein, 30 aa, Lama glama
QVQLQESGPGLVKPSQTLSLTCTISGVSIT

SEQ ID NO: 43 - protein, 14 aa, Lama glama
WIRQAPGKGLEWMG

SEQ ID NO: 44 - protein, 14 aa, Lama glama
WIRQPPGKGLEWMG

SEQ ID NO: 45 - protein, 14 aa, Lama glama
WIRQPPGKGLEWMG

SEQ ID NO: 46 - protein, 14 aa, Lama glama
WIRQPPGKGLEWMG

SEQ ID NO: 47 - protein, 14 aa, Lama glama
WIRQPPGKGLDWMG

SEQ ID NO: 48 - protein, 14 aa, Lama glama
WIRQSPGKGLEWMG

SEQ ID NO: 49 - protein, 14 aa, Lama glama
WIRQPPGKGLEWMG

SEQ ID NO: 50 - protein, 14 aa, Lama glama
WIRQPPGKGLEWMG

SEQ ID NO: 51 - protein, 14 aa, Lama glama
WIRQPPGKGLEWMG

SEQ ID NO: 52 - protein, 14 aa, Lama glama
WIRQPPGKGLEWMG

SEQ ID NO: 53 - protein, 14 aa, Lama glama
WIRQPPGKGLEWMG

SEQ ID NO: 54 - protein, 14 aa, Lama glama
WIRQPPGKGLEWMG

SEQ ID NO: 55 - protein, 14 aa, Lama glama
WIRQPPGKGLEWMG

SEQ ID NO: 56 - protein, 14 aa, Lama glama
WIRQPPGKGLEWMG

SEQ ID NO: 57 - protein, 14 aa, Lama glama
WIRQPPGKGLEWMG

SEQ ID NO: 58 - protein, 14 aa, Lama glama
WIRQPPGKGLEWMG

SEQ ID NO: 59 - protein, 32 aa, Lama glama
RTSISRDTSKNQFSLQLGSVTPEDTAVYYCAQ

SEQ ID NO: 60 - protein, 32 aa, Lama glama
RTSISRDTSKNQFSLQLSSVTPEDTAVYYCAR

SEQ ID NO: 61 - protein, 32 aa, Lama glama
RTSISRDTSKNQFSLQLSSVTPEDTAVYYCGR

SEQ ID NO: 62 - protein, 32 aa, Lama glama; identical to SEQ ID NO: 60

SEQ ID NO: 63 - protein, 32 aa, Lama glama
RTSISRDTSRNQFSLQLSSVTPTDTAVYYCAR

SEQ ID NO: 64 - protein, 32 aa, Lama glama
RTSISRDTSKNHFTLQLTSVTPEDTAVYYCAR

SEQ ID NO: 65 - protein, 32 aa, Lama glama; identical to SEQ ID NO: 9

SEQ ID NO: 66 - protein, 32 aa, Lama glama
RTSISRDTSKNHFTLHLSSLTPEDTAVYYCAR

SEQ ID NO: 67 - protein, 32 aa, Lama glama; identical to SEQ ID NO: 60

SEQ ID NO: 68 - protein, 32 aa, Lama glama
QTSISRDTSKNQFSLHLSSVTPEDTAVYYCAR

SEQ ID NO: 69 - protein, 32 aa, Lama glama
RTSISRDTSKNQFTLQLSSVTPEDTAVYYCAG

SEQ ID NO: 70 - protein, 32 aa, Lama glama
RTSISRDTSQNHFTLQLSSVTPEDTAVYYCAR

SEQ ID NO: 71 - protein, 32 aa, Lama glama
RASISRDTSKNRFTLQVSSVTPEDTAVYYCAR

SEQ ID NO: 72 - protein, 32 aa, Lama glama
RTSISRDTSKNQFTLQLNSVTPEDTAAYYCAR

SEQ ID NO: 73 - protein, 32 aa, Lama glama; identical to SEQ ID NO: 9

SEQ ID NO: 74 - protein, 32 aa, Lama glama
RTSISRDTSKNQFTLQLTSVTLEDTAVYYCAR

SEQ ID NO: 75 - protein, 11 aa, Lama glama
WGKGTLVTVSS

SEQ ID NO: 76 - protein, 11 aa, Lama glama
WGKGTLVTVSS

SEQ ID NO: 77 - protein, 11 aa, Lama glama
WGQGTQVTVSS

SEQ ID NO: 78 - protein, 11 aa, Lama glama
WGQGTQVTVSS

SEQ ID NO: 79 - protein, 11 aa, Lama glama
WGQGTLVTVSS

SEQ ID NO: 80 - protein, 11 aa, Lama glama
WGQGTLVTVSS

SEQ ID NO: 81 - protein, 11 aa, Lama glama
WGQGTQVTVSS

SEQ ID NO: 82 - protein, 11 aa, Lama glama
WGQGTQVTVSS

SEQ ID NO: 83 - protein, 11 aa, Lama glama
WGQGTQVTVSS

SEQ ID NO: 84 - protein, 11 aa, Lama glama
WGQGTQVTVSS

SEQ ID NO: 85 - protein, 11 aa, Lama glama
WGQGILVTVSS

SEQ ID NO: 86 - protein, 11 aa, Lama glama
WGQGTQVTVSS

SEQ ID NO: 87 - protein, 11 aa, Lama glama
WGKGTLVTVSS

SEQ ID NO: 88 - protein, 11 aa, Lama glama
WGQGTQVTVSS

SEQ ID NO: 89 - protein, 11 aa, Lama glama
WGQGTQVTVSS

SEQ ID NO: 90 - protein, 11 aa, Lama glama
WGQGTQVTVSS

SEQ ID NO: 91 - DNA, 51 bp, Artificial Sequence
  primer
ggtgggagctcacagctctgtcctcacacagggctgaggtcgctggggaag

SEQ ID NO: 92 - DNA, 57 bp, Artificial Sequence
  primer
atgagactgttgggtcttctcctgtgcctggtggcaggtccccaaggtgtcctgtcc

SEQ ID NO: 93 - DNA, 21 bp, Artificial Sequence
  primer
acagctctgtcctcacacagg

SEQ ID NO: 94 - DNA, 23 bp, Artificial Sequence
  primer
agaggaagatgagactgttgggt

SEQ ID NO: 95 - protein, 19 aa, Lama glama
MRLLGLLLCLMAGPQGVLS

SEQ ID NO: 96 - protein, 19 aa, Lama glama
MRLLGLLLCLVVGPQGVLS

SEQ ID NO: 97 - protein, 19 aa, Lama glama
MRLLGLLLCLVAGPQGVLS

SEQ ID NO: 98 - DNA, 21 bp, Artificial Sequence
  Primer
acagctctgtcctcacacagg

SEQ ID NO: 99 - DNA, 19 bp, Artificial Sequence
  Primer
ccagctccaagtgtcccaa

SEQ ID NO: 100 - DNA, 52 bp, Artificial Sequence
  Primer
tagttctaaacggcccagccggccatggcccaggtgcagctgcaggagtcgg

SEQ ID NO: 101 - DNA, 18 bp, Artificial Sequence
  Primer
tgaggagacggtgacctg

SEQ ID NO: 102 - protein, 125 aa, Lama glama
QVQLQESGPGLVKPSQTLSLTCTVSGGSITTYRYYWSWIRQPPGKGLEWMGAIAYSGSTYYSPSLKSRTSISRDTTKNQFTLQLSSVTPEDTAVYYCARGRLGSWYYELNEYDYWGQGTQVTVSS

SEQ ID NO: 103 - protein, 125 aa, Lama glama; identical to SEQ ID NO: 102

SEQ ID NO: 104 - protein, 126 aa, Lama glama
QVQLQESGPGLVKPSQTLSLTCTVSGGSITTYNYAWSWIRQPPGKGLEWMGVIAYDGNTYYSPSLKSRTSISRDTSKNQFSLQLSSVTPEDTAVYYCARGTVGSWYDEFPPRYDYWGQGTQVTVSS

SEQ ID NO: 105 - protein, 126 aa, Lama glama; identical to SEQ ID NO: 104

SEQ ID NO: 106 - protein, 126 aa, Lama glama; identical to SEQ ID NO: 104

SEQ ID NO: 107 - protein, 126 aa, Lama glama
QVQLQESGPGLVKPSQTLSLTCTVSGGSITTYNYAWSWIRQPPGKGLEWMGVIAYDGSTYYSPSLKSRTSISRDTSKNQISLRLSSVTPEDTAVYYCARGTVGSWYDEFPPRYDYWGQGTQVTVSS

SEQ ID NO: 108 - protein, 126 aa, Lama glama
QVQLQESGPGLVKPSQTLSLTCTVSGGSITTYNYAWSWIRQPPGKGLEWMGVIAYDGSTYYSPSLKSRTSISRDTSKNQFSLQLSSVTPEDTAVYYCARGTVGSWYDEFPPRYDYWGQGTQVTVSS

SEQ ID NO: 109 - protein, 126 aa, Lama glama; identical to SEQ ID NO: 108

SEQ ID NO: 110 - protein, 126 aa, Lama glama
QVQLQESGPGLVKPSQTLSLTCTVSGGSITTYNYAWTWIRQPPGKGLEWMGVMAYDGSTYYSPSLKSRTSISRDTSKNQFSLQLRSATPEDTAVYYCARGTVGSWYDEFPPRYDYWGQGTQVTVSS

SEQ ID NO: 111 - protein, 126 aa, Lama glama
QVQLQESGPGLVKPSQTLSLTCTVSGGSITTYNYAWSWIRQPPGKGLEWMGVIAYDGSTYYSPSLKSRASISRDTSKNQFSLQLSSVTPEDTAVYYCARGTVGSWYDEFPPRYDYWGQGTQVTVSS

SEQ ID NO: 112 - protein, 125 aa, Lama glama
QVQLQESGPGLVKPSQTLTLTCTVSGDSITTNYYYWSWIRQPPGKGLEWMGTIDYSGRTYYSPSLKSRASVSRDTSKDQFTLQLTSVTPEDTAVYYCARASLIKVVHGKDEYNAWGHGTQVTVSS

SEQ ID NO: 113 - protein, 125 aa, Lama glama
QVQLQESGPGLVKPSQTLSLTCTVSGGSITTYRYYWSWIRQPPGKGLEWMGAIAYSGSTYYSPSLKSRTSMSRDTTKNQFTLQLSSVTPEDTAVYYCARGRLGSWYYELNEYDYWGQGTQVTVSS

SEQ ID NO: 114 - protein, 125 aa, Lama glama; identical to SEQ ID NO: 102

SEQ ID NO: 115 - protein, 125 aa, Lama glama; identical to SEQ ID NO: 102

SEQ ID NO: 116 - protein, 125 aa, Lama glama; identical to SEQ ID NO: 102

SEQ ID NO: 117 - protein, 125 aa, Lama glama; identical to SEQ ID NO: 102

SEQ ID NO: 118 - protein, 125 aa, Lama glama; identical to SEQ ID NO: 102

SEQ ID NO: 119 - protein, 125 aa, Lama glama; identical to SEQ ID NO: 102

SEQ ID NO: 120 - protein, 125 aa, Lama glama; identical to SEQ ID NO: 102

SEQ ID NO: 121 - protein, 125 aa, Lama glama; identical to SEQ ID NO: 102

SEQ ID NO: 122 - protein, 98 aa, Lama glama
QVQLQESGPGLVKPSQTLSLTCTVSGGSITTSYYYWSWIRQPPGKGLEWMGAIYSGSTYYSPSLKSRTSISRDTSNNQFSLQLSSVTPEDTAVYYCAR

SEQ ID NO: 123 - protein, 98 aa, Lama glama
QVQLQESGPGLVKPSQTLSLTCTVSGGSITTSYYYWSWIRQPPGKGLEWMGAIYSGSTYYSPSLKSCTSISRDTSNNQFSLQLSSVTPEDTAVYYCAR

SEQ ID NO: 124 - protein, 99 aa, Lama glama
QVQLQEWGPGLLKPSQTLSLTCAVYGGSITTSYYYWSWIRQPPGKGLEWMGVIGYEGSTYYSPSLKSHTSISRDTSKNQFSLQLSSVTPEDTAVYYCAR

SEQ ID NO: 125 - protein, 99 aa, Lama glama
QVQLQEWGPGLLKPSQTLSLTCAVYGGSITTSYYYWSWIRQPPGKGLEWMGVIGYEGSTYYSPSLKSRTSISRDTSKNQFSLQLSSVTPEDTAVYYCAR

SEQ ID NO: 126 - protein, 99 aa, Lama glama
QVQLQESGPGLVKPSQTLSLTCTVSGGSITTSYYAWSWIRQPPGKGLEWMGVIAYDGSTYYSPSLKSRTSISRDTSKNQFSLQLSSVTPEDTAVYYCAR

SEQ ID NO: 127 - protein, 99 aa, Lama glama
QVQLQESGPGLVKPSQTLSLTCTVSGGSITTNYYYWSWIRQPPGKGLEWMGAIAYSGSTYYSPSLKSRTSISRDTSKNQFTLQLSSVTPEDTAVYYCAR

SEQ ID NO: 128 - protein, 99 aa, Lama glama
QVQLQESGPGLVKPSQTLSLTCTVSGGSITTNYYYWSWIRQPPGKGLEWMGAIAYSGSTYYSPSLKSRTSISRDTSKNQFSLQLSSVTPEDTAVYYCAR

SEQ ID NO: 129 - protein, 98 aa, Lama glama
QVQLQESGPGLVKPSQTLSLTCAVYGGSITTSCYAWSWICQPPEKGLEWMAAIYSGSTYYSPSLKSHTSISRDMSKNQFSLQLSSVTPEDTAVYYCAR

SEQ ID NO: 130 - protein, 97 aa, Lama glama
QVQLQESGPGLVKPSQTLSLTCTVSGGSITTSCYAWSWIHQPPGKGLEMGAIYSGSTYYSPSLKSHTSISRDTSKNQFSLQLSSVTPEDTAVYYCAR